(19) United States
(12) Patent Application Publication
(10) Pub. No.: US 2026/0110004 A1
(43) Pub. Date: Apr. 23, 2026

(54) PROCESS FOR PREPARING LACTIC ACID

(71) Applicant: CARGILL, INCORPORATED, Wayzata, MN (US)

(72) Inventors: Emily Marie Schmitz Bunch, Omaha, NE (US); Christopher Lawrence Frank, Oskaloosa, IA (US); Nolan Ray Mente, Pella, IA (US); Joseph Spencer, Bennington, NE (US); Eric Stanley Sumner, Minneapolis, MN (US)

(73) Assignee: CARGILL, INCORPORATED, Wayzata, MN (US)

(21) Appl. No.: 19/482,324

(22) PCT Filed: May 10, 2024
(86) PCT No.: PCT/US2024/028717

§ 371 Date: Nov. 7, 2025

Related U.S. Application Data
(60) Provisional application No. 63/465,667, filed on May 11, 2023.

(51) Int. Cl. C12P 7/56; B01D 21/26; B04B 7/08; B04C 9/00; B04C 11/00

(52) U.S. Cl. CPC C12P 7/56; B01D 21/262; B01D 21/267; B04B 7/08; B04C 9/00; B04C 11/00; B04C 2009/002

(57) ABSTRACT

Lactic acid is prepared by fermenting a carbohydrate source in the presence of a calcium hydroxide-based pH control agent that comprises from about 0.1 to about 10 wt % silica to form a fermentation broth comprising lactic acid and calcium lactate. The fermentation broth is processed by a sequence of hydrocyclones to remove silica from the feed streams to avoid fouling of equipment and to avoid damage to filtration or centrifugation equipment. The biomass component of the fermentation broth is separated out prior to acidulation of the fermentation broth, and therefore itself is a viable product for use since it can be provided without filter aid components that were previously required when the biomass was removed during acidulation. Gypsum is recovered after the acidulation step, and can be available as a product by itself, or combined with the biomass as a mixed product.

### CROSS-REFERENCE TO RELATED APPLICATIONS

This application claims the benefit of U.S. Provisional Application No. 63/465,667, filed May 11, 2023, which is incorporated by reference herein in its entirety.

### FIELD

The present invention relates to preparation and recovery of lactic acid products and lactic acid. The present invention also relates to preparation and recovery of other hydroxyacids produced by fermentation such as 3-hydroxypropanoic acid (3HP), 3-hydroxybutyric acid, 4-hydroxybutyric acid, 3-hydroxyhexanoic acid, 3-hydroxyoctanoic acid, 3-hydroxydecanoic acid, and the like.

### BACKGROUND

A process for preparing a purified lactic acid solution is described in U.S. Pat. No. 7,026,145, which includes the steps of providing a source of lactate material which includes a calcium salt; acidulating the concentrated broth with sulfuric acid to form an acidulated solution which includes lactic acid and calcium sulfate; reducing an amount of calcium sulfate from the acidulated solution; extracting the acidulated solution with an amine extractant to form a loaded solvent; and back extracting the loaded solvent with an aqueous solvent to provide a purified solution of lactic acid.

A method for separating biomass from solid fermentation product is described in WO 2017/207501, wherein a slurry comprising biomass and solid fermentation product is provided to the top of a biomass separator unit and an aqueous medium is provided to the bottom of a biomass separator unit, while a product stream comprising solid fermentation product is withdrawn from the bottom of the biomass separator unit and a waste stream comprising biomass is withdrawn from the top of the biomass separator unit.

WO 2004/038032 describes processes to separate the biomass from the lactic acid-containing fermentation product by a) subjecting the fermentation broth to an alkalifying step, b) adding one or more flocculants, and c) separating the biomass floes from the lactate/lactic acid-containing fermentation broth.

### SUMMARY

Lactic acid products (e.g., containing lactic acid and/or lactate anions (as further described below) advantageously can be prepared by fermenting monomeric saccharides (for example glucose and/or fructose) and/or oligomeric polysaccharide (for example, sucrose, maltose, isomaltose, hydrolyzed starch products and starches) sources using, for example, yeasts, filamentary fungi, or bacteria (hereinafter sometimes referred to as the “fermenting organism”). Preferably, starch hydrolysates containing between 50 wt % and 99 wt % glucose (based on the dry solids content of the hydrolysate) are utilized, for example, starch hydrolysates containing from 60 wt % to 97 wt % glucose (in some instances starch hydrolysates containing from 80 wt % to and 96 wt % glucose (based on the dry solids content of the hydrolysate). In an alternative aspect, the starch hydrolysate added comprises between 25 wt % and 80 wt % glucose (for example 30 wt % to 75 wt %), and preferably 30 wvt % to70 wt % glucose based on the dry solids content. In this alternative embodiment, a glucoamylase is added to generate glucose from the oligomers and polymers of glucose contained within the starch hydrolysate for use by the fermenting organism during the fermentation. Typically, the organism is allowed to ferment with the initial starch hydrolysate introduced at the start of the fermentation until the glucose concentration in the fermentation broth is 3 weight percent or less. Then, as the fermentation continues, additional hydrolysate is introduced while maintaining the glucose concentration at less than 3 wt % based on the wet weight of the fermentation broth, preferably less than 2 wt %, and in some instances less than 1.5 wt % based on the wet weight of the fermentation broth. For example, as the additional hydrolysate is being fed (during a fed batch fermentation), the hydrolysate is introduced at a rate that typically will maintain the glucose concentration from 1 wt % to 1.5 wt % (e.g., from 10 g/L to 15 g/L) of the fermentation broth. Additionally details of this alternative embodiment is disclosed in PCT/US2021/013559 dated 17 Jan. 2020 (published 22 Jul. 2021 as WO 2021/146509 A1), which is hereby incorporated by reference herein.

During the fermentation process, it may be desirable to adjust the pH of the fermentation broth to enhance the activity of the fermenting organism (e.g., maintain the pH at a level where the performance of the fermenting organism and the efficiency of producing lactic acid products is optimized). When desired, a basic compound (sometimes hereinafter referred to as a ′pH control agent”) typically would be added to the fermentation broth during the fermentation to adjust the pH of the broth to a pH suitable for the organism being utilized.

While a number of basic compounds could be used, it has been found that calcium hydroxide and/or calcium oxide pH control agents (collectively referred to as calcium hydroxide-based pH control agents), such as lime is economical and easy to use to raise the pH of the broth (and/or maintain the pH at a desired level as lactic acid products are being produced). The pH of the broth is typically maintained for a desired period of time at a pH of from 2.5 to 5.0 during the fermentation (for example, from 2.8 to 4.5 or from 3.0 to 4.2) and the pH at the end of the fermentation is typically from 2.5 to 4.0 (for example from 2.7 to 3.5). At these pH's aqueous solutions containing the lactic acid products typically comprise both lactic acid and the anion of a monomeric lactate salt in solution. In instances where the concentration of lactic acid products is particularly high during the fermentation (for example lactic equivalents greater than 125 gram/liter or greater than 130 gram/liter and the pH is greater than 3.5 or greater than 4.0), a small portion of the monomeric lactate salt (e.g., calcium lactate) may be insoluble. However, as the pH is lowered (in the fermenter or downstream of the fermenter) and/or the temperature is raised downstream of the fermenter, the insoluble monomeric lactate salt will resolubilize. The pH of the lactic acid product and the temperature of the aqueous solutions containing the lactic acid products from the end of the fermentation (the fermentation broth) through the solution present during the recovery steps are maintained in appropriate ranges to maintain substantially all the lactic acid and lactic acid salts in solution. For example, the pH typically is in the range of from 2.5 to 4.0 (for example from 2.7 to 3.5) after the end of fermentation and prior to the acidulation step described below; and, after the end of fermentation, the temperature typically is at least 45° C. at least 50° C., at least 55° C., and preferably at least 60° C.;, and, except for during an evaporation/concentration step, typically in the range of from 45° Celsius to 90° Celsius (for example from 50° Celsius to 85° Celsius, preferably from 60° Celsius to 80° Celsius) after the end of fermentation and prior to the acidulation step. During an evaporation/concentration step where the lactic acid product concentration is being increased, the temperature typically is from 50° C. to 150° C., 60° C. to 140° C., 70° C. to 135° C., or 80° C. to 100° C., and in a particular preferred embodiment where higher vacuum is utilized, from 50° C. to 90° C., (preferably from 70° C. to 85° C., and more preferably 70° C. to 80° C.).

In principle, the final lactic acid is easy to obtain in relatively pure form in solutions obtained from a fermentation utilizing calcium hydroxide-based pH control agents through the acidulation of an aqueous solution containing the lactic acid product with sulfuric acid. The sulfuric acid lowers the pH of the solution and therefore converts the lactate salt anions to free lactic acid. The calcium and sulfate ions react to form calcium sulfate (gypsum). The gypsum precipitates from the aqueous solution and can be readily separated from the lactic acid product (substantially free lactic acid), which remain in the aqueous solution. This provides a ready way to use a higher pH during the fermentation to improve the performance of the organism in making lactic acid products (i.e., increased rate, titer and yield of lactic acid products from the carbon feedstock), and also provides a ready way to separate the ions, such as sulfate and calcium, from the solution containing the lactic acid. A process for producing lactic acid products that utilizes a calcium hydroxide-based pH control agent sulfuric acid for acidification and the removal of gypsum, is sometimes referred to as a gypsum removal process. The remaining lactic acid containing solution can be further purified (for example, to remove undesirable cation and anions) and the lactic acid concentration can be increased by methods known to one of skill in the art, such as ion exchange, evaporation, and distillation.

Production and recovery of lactic acid products obtained from fermentation using a gypsum removal process can be readily carried out without incident on the lab scale or small batch production. However, it has been discovered that calcium hydroxide-based pH control agents, such as lime, frequently contain an undesirable amount of silica and that the amount of silica frequently contained in lime surprisingly causes severe damage to filtration and/or centrifugation equipment that would be used in large scale production facilities to remove biomass prior to acidulation. Processes for producing lactic acid through fermentation using a gypsum removal process and that also remove biomass in a step prior to the acidulation will either suffer from equipment failure in the biomass removal equipment or suffer large yield losses, depending on the process steps used.

Similar processes are used for the recovery of other hydroxyacids, and similar concerns apply when gypsum removal processes and biomass removal are utilized.

A continuous process for preparing lactic acid has been discovered utilizing unique steps and equipment that efficiently provides lactic acid products in large production scale quantities and provides lactic acid products that can be readily recovered as lactic acid. For purposes of this document “recovering” or “recovery” of lactic acid refers to the process steps downstream of fermentation (i.e., after the fermentation). And, for the purposes of this document, “continuous” refers to a process step or steps where the step or steps are carried out continuously typically for at least four (4) days, and preferably at least five (5), at least six (6), and at least seven (7) days. For clarification, a recovery process may be carried out continuously (e.g., at least four (4) days), while each individual step/unit operation not occurring simultaneously. For example, surge tanks and storage volumes may be installed at the end of a unit operation (or processing step) that enables the recovery process to be carried out continuously, while allowing any particular unit operation (or processing step) to be idled and/or paused while the remaining recovery steps continue to be carried out. Additionally, an alternative unit operations or equipment could be provided to use while another unit operation or piece of equipment is taken offline for maintenance, cleaning, calibration or repair. Examples of situations where the idling of a unit operation may be beneficial are for maintenance, cleaning, calibration, and repair of the particular equipment being used, and other reasons that would be apparent to one of ordinary skill in the art. “Recovered” refers to the relatively pure aqueous lactic acid that is finally obtained when all the processing is completed.

In an aspect, a process for preparing lactic acid comprises
i) fermenting a carbohydrate source in the presence of a calcium hydroxide-based pH control agent that comprises from about 0.1 to about 10 wt %, silica (typically from 2.5 to 7.5 wt %) to form a fermentation broth comprising lactic acid and calcium lactate:
ii) continuously directing the fermentation broth to a primary hydrocyclone at a temperature above a temperature at which the calcium lactate is substantially soluble (i.e., at least 90 wt % soluble by weight, preferably at least 95 wt %, and more preferably at least 99 wt % soluble by weight), and withdrawing from the primary hydrocyclone:
   a) a primary hydrocyclone overflow stream comprising lactic acid, calcium lactate, biomass, and water, wherein the primary hydrocyclone overflow stream contains no more than about 0.05 wt % of silica, and
   b) a primary hydrocyclone underflow stream comprising water enriched in silica, and further comprising biomass, lactic acid, and calcium lactate.

The primary hydrocyclone underflow stream typically comprises both solid particles (e.g., silica and biomass) and liquid components (typically comprising water and lactic acid product. The primary hydrocyclone underflow stream is continuously directed to a secondary hydrocyclone (preferably, separate from the primary hydrocyclone), which in turn has a secondary hydrocyclone overflow stream and hydrocyclone underflow stream withdrawn therefrom. The secondary hydrocyclone overflow stream comprises lactic acid, calcium lactate, biomass, and water, wherein the secondary hydrocyclone overflow stream contains no more than 0.05 wt % of silica. The secondary hydrocyclone underflow stream comprising water enriched in silica, and also comprising some biomass, lactic acid and calcium lactate.

For purposes of the present discussion, a stream is enriched in a component if it contains a higher concentration of the identified component than the concentration of the identified component before it was acted upon. For example, the secondary hydrocyclone underflow stream is enriched in silica because it contains a higher concentration of silica than the concentration of silica in the stream that was initially provided to the inlet of the secondary hydrocyclone.

The primary hydrocyclone overflow stream and the secondary hydrocyclone overflow stream are directed to a centrifuge.

The centrifuge acts upon the incoming streams to produce two outlet streams, a clarified broth stream containing water, lactic acid and soluble calcium lactate, and a centrifuge concentrate stream enriched in biomass.

The clarified broth stream is directed to an acidulation station, where it is acidulated with sulfuric acid to form an aqueous lactic acid product and insoluble components comprising gypsum. The insoluble components comprising gypsum are then separated from the aqueous lactic acid product. The lactic equivalents in the aqueous lactic acid product comprises at least 80 weight percent lactic acid based on the total lactic acid product present, preferably at least 90 weight percent lactic acid based on the total lactic acid product present, and more preferably at least 95 weight percent lactic acid based on the total lactic acid product present (for example, from 97-98 weight percent lactic acid based on the total lactic acid product present). Lactic acid refers to lactic acid that is not associated with a cation, such as calcium.

In another aspect, a process for preparing lactic acid comprises
i) fermenting a carbohydrate source in the presence of a calcium hydroxide-based pH control agent that comprises from about 0.1 to about 10 wt % silica (typically from 2.5 wt % to 7.5 wt %) to form a fermentation broth comprising lactic acid and substantially soluble (i.e., at least 90% soluble, preferably at least 95% soluble, and more preferably at least 99% soluble by weight) calcium lactate;
ii) directing the fermentation broth to a primary hydrocyclone at a temperature above a temperature at which the calcium lactate is substantially soluble (i.e., at least 90% soluble, preferably at least 95 wt %, and more preferably at least 99% soluble by weight), and withdrawing from the primary hydrocyclone:
   a) a primary hydrocyclone overflow stream comprising lactic acid, calcium lactate, biomass, and water, wherein the primary hydrocyclone overflow stream contains no more than about 0.05 wt % of silica, and
   b) a primary hydrocyclone underflow stream enriched in silica and comprising water, biomass, lactic acid and calcium lactate.

The primary hydrocyclone underflow stream comprises both solid particles (e.g., silica) and liquid components (typically, comprising water and lactic acid product). The primary hydrocyclone underflow stream is directed to a secondary hydrocyclone, which in turn has a secondary hydrocyclone overflow stream and hydrocyclone underflow stream withdrawn therefrom. The secondary hydrocyclone overflow stream comprises lactic acid and calcium lactate, biomass, and water, wherein the secondary hydrocyclone overflow stream contains no more than 0.05 wt % silica. The secondary hydrocyclone underflow stream is enriched in silica and comprises water, biomass, lactic acid, and calcium lactate.

The primary hydrocyclone overflow stream and the secondary hydrocyclone overflow stream are directed to a centrifuge, which in turn produces a clarified broth stream comprising lactic acid and calcium lactate, and a centrifuge concentrate stream enriched in biomass.

The secondary hydrocyclone underflow stream and the centrifuge concentrate stream enriched in biomass are directed to a decanter/centrifuge as a decanter/centrifuge biomass feed having a combined dry substance content of from about 1 to about 30 wt %.

The decanter/centrifuge in turn produces a decanter/centrifuge centrate enriched in lactic acid and calcium lactate, and a decanter/centrifuge underflow stream enriched in biomass having a dry substance content of at least about 30 wt %, preferably at least 35 wt %.

The clarified broth stream and the decanter/centrifuge centrate are directed to an acidulation station and acidulated with sulfuric acid to form an aqueous lactic acid product and insoluble components comprising gypsum. The insoluble components comprising gypsum are then separated from the aqueous lactic acid product. The lactic equivalents in the aqueous lactic acid product comprises at least 80 wt % lactic acid based on the total lactic acid product present, preferably at least 90 wt % lactic acid based on the total lactic acid product present, and more preferably at least 95 wt % lactic acid based on the total lactic acid product present (for example, from 97-98 wt % lactic acid based on the total lactic acid product present). Lactic acid refers to lactic acid that is not associated with a cation, such as calcium.

Other configurations of the present process are described in more detail below. For example, typically and preferably the fermentation broth preferably is evaporated to increase the concentration of lactic acid equivalents in the fermentation broth before it is directed to the hydrocyclones, as described below. Concentrating the lactic acid product before it enters the hydrocyclones, is beneficial in reducing the amount of hydrocyclones necessary.

Processes as described herein provide particular benefits. In an aspect, by removing biomass prior to acidulation, the biomass itself becomes a viable product for use since it can be provided without filter aid components that were previously required when the biomass was removed during or after acidulation. Additionally, the by-product of the acidulation step of lactic acid product (lactic and calcium lactate) with sulfuric acid is gypsum, which is a soft sulfate mineral composed of calcium sulfate dihydrate, with the chemical formula CaSQ4·2H2O. Gypsum is widely mined and is used as a fertilizer, and therefore can be a commercially valuable product in itself.

Advantageously, the processes as described herein can be configured to provide the biomass as a product without gypsum. And, advantageously, a gypsum product can be provided as a product substantially free of biomass. In an aspect, processes as described herein can be configured to combine the biomass with the gypsum after the acidulation step to provide a combined biomass/gypsum product.

It has been found that by removing biomass prior to acidulation, production rates can be substantially increased using the same filter systems. For example, if used, filter aid consumption at post acidulation filters, such as drum filters can be substantially reduced, and production rates can be substantially increased. In an aspect, process configurations as described herein can provide additional benefits, such as reduction in process cation resin fouling and lateral plugging of feed lines, and providing a gypsum product that is drier than usually obtained in prior art lactic acid production processes.

Additionally, it has been found that removal of silica early in the process by use of hydrocyclones in the manner described herein not only prevents or substantially decreases damage to the filtration or centrifugation equipment, the early removal of silica in the process facilitates effective and efficient centrifugal biomass separation, which in turn improves gypsum separation and allows for increased lactic acid recovery rates.

Typically, the fermentation step is carried out in a batch or fed-batch mode, while the recovery step(s) typically are carried out using continuous modes. The step or steps of the recovery are typically carried out continuously typically for at least four (4) days, at least five (5) days, at least six (6) days, preferably at least seven (7) days, more preferably the recovery step or steps are carried out continuously for at least fourteen (14) days, and in some instances at least thirty (30) days. As discussed above, the recovery process may be carried out in a continuous manner, while particular unit operations being idled at times while the overall process is carried out continuously.

### BRIEF DESCRIPTION OF THE DRAWINGS

The accompanying drawings, which are incorporated in and constitute a part of this application, illustrate several aspects of the invention and together with a description of the embodiments serve to explain the principles of the invention. A brief description of the drawings is as follows:

FIG. 1 is a schematic process flow diagram of an aspect of the process as described herein.

FIG. 2 is a schematic process flow diagram of an aspect of the process as described herein.

FIG. 3 is a schematic process flow diagram of an aspect of the process as described herein.

FIG. 4 is a schematic process flow diagram of an aspect of the process as described herein.

FIG. 5 is a schematic process flow diagram of an aspect of the process as described herein.

FIG. 6 is a schematic process flow diagram of an aspect of the process as described herein.

FIG. 7 is a schematic process flow diagram of an aspect of the process as described herein.

FIG. 8 is a schematic process flow diagram of an aspect of the process as described herein.

FIG. 9 is a schematic process flow diagram of the lactic acid separation process used in Example 1 for comparison to the methods described herein.

FIG. 10 shows silica content in samples from four different process streams of process 100 as described in Example 2.

FIG. 11 shows total suspended solids from samples as outlined in Example 2.

### DETAILED DESCRIPTION

The aspects of the present invention described below are not intended to be exhaustive or to limit the invention to the precise forms disclosed in the following detailed description. Rather, a purpose of the aspects chosen and described is by way of illustration or example, so that the appreciation and understanding by others skilled in the art of the general principles and practices of the present invention can be facilitated.

In the present discussion, various streams or compositions are described as comprising calcium lactate or lactic acid, and it is understood that in such streams or compositions calcium lactate and lactic acid may both be present in equilibrium. For purposes of the present discussion, lactate salts and lactic acid in an aqueous liquid are referred to generically as lactic acid products. The fermentation broth and subsequent streams and compositions in the process prior to acidulation comprise both calcium lactate salts (in solution) and lactic acid, in individual percentages determined by the pH of the stream(s), while after acidulation (i.e. a pH of from 1.4 to 2.5, and preferably from 1.5 to 2.0), lactic acid product in the streams and composition in the process comprise predominantly or completely lactic acid. For purposes of the present discussion, the concentration of calcium lactate and/or of lactic acid in any given composition (such as the lactic acid products) is measured on the basis of the total amount of lactic functionality present (whether in the acid form and/or in the form of an anion of a lactate salt in solution), and expressed in terms of “lactic equivalents”, or “lactic equivalents concentration.” For clarification, the values identified as lactic equivalents (and lactic equivalents concentration) do not include the weight of any cation of the lactate salt that may be present in the solution. For example, the lactic equivalent for an aqueous solution would not include the calcium that may be present in the solution.

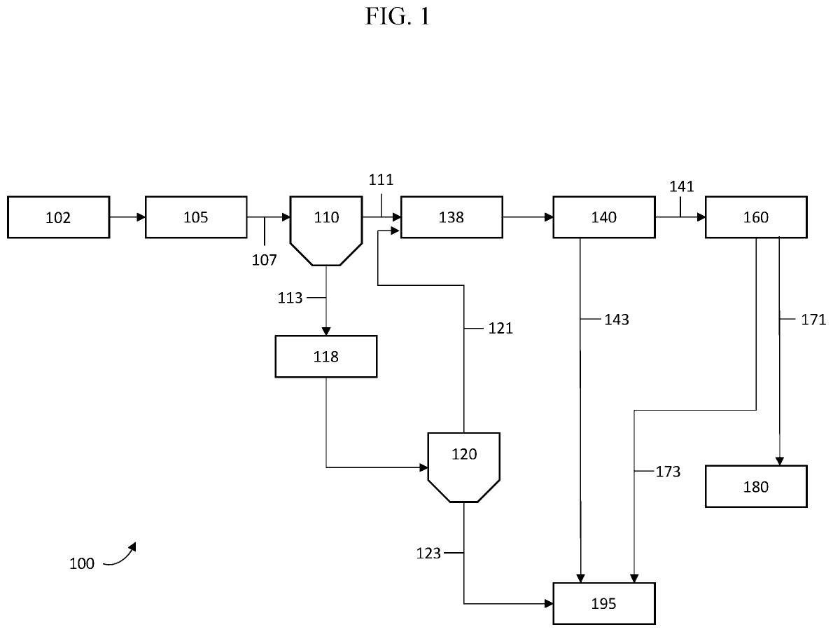

Turning now to the Figures, FIG. 1 is a schematic process flow diagram of an aspect of a process 100 for preparing lactic acid product. In this process, a carbohydrate source is fermented in fermenter 102.

Fermentation can be conducted using microorganisms such as bacteria, fungi or yeast, capable of forming lactic acid product (lactic acid and/or lactate) upon metabolizing a carbon source. Such lactic acid and/or lactate material producing organisms are known. For bacteria, typically the family Lactobacillaceae are employed. As to fungi, those of the family Rhizopus can be employed. Suitable yeast include yeast of the genus Saccharomyces. Issatchenkia, and Kluveromyces, for example, Issatchenkia orientalis Saccharomyces cerevisiae, and Kluveromyces [marxionis Fill in Myrah}). The most preferred organism utilized for the production of lactate and lactic acid are genetically modified Issatchenkia orientalis that have been modified to produce lactic acid. Examples of genetically modified Issatchenkia orientalis are described in U.S. Pat. No. 8,097,448 B2 issued Jan. 17, 2012 to Suominen et al. entitled “Genetically Modified Yeast Of The Species Issatchenkia Orientalis And Closely Related Species, And Fermentation Processes Using Same”, which is incorporated by reference herein for its teachings regarding such organisms. Issatchenkia orientalis may also be referred to in the art as Pichia kudriavzevii, Candida krusei, and/or Candida glycerinogenes, see for example Douglass A. P. et al. “Population genomics shows no distinction between pathogenic Candida krusei and environmental Pichia kudriavaevii: One species, four names,” Jul. 19, 2018. PLOS Pathogens. Fermentation temperatures, conditions, and nutrient media for preparation of lactic acid products are generally known.

As discussed above, a calcium hydroxide-based pH control agent is added to the fermentation broth to adjust the pH of the broth and therefore enhance the activity of the organism being utilized. Typically, the pH of the fermentation broth during fermentation is adjusted to from about 2.5 to about 5.0. Preferably, the pH of the fermentation broth is maintained at a pH of from 2.8 to 4.5 (or from 3.0 to 4.2) by adding calcium-hydroxide-based pH control agent and maintained within that range until a sufficient time has been achieved so that the addition of calcium hydroxide-based pH control agent can be ceased, and thereafter the fermentation can be continued until the desired end of fermentation pH of from 2.5 to 4.0 (for example from 2.7 to 3.5) is obtained. At these pH's both lactic acid and calcium lactate are present in the fermentation broth.

The pH and temperature of the solutions containing the calcium lactate and lactic acid during the fermentation and recovery steps are maintained in appropriate ranges to maintain substantially all the lactic acid and calcium lactate (i.e., at least 90% and preferably at least 95%) in solution. For example, the pH typically is in the range of from 2.5 to 4.0 (for example from 2.7 to 3.5) after the end of fermentation and prior to the acidulation step described below; and, after the end of fermentation, the temperature typically is at least 45° C. at least 50° C. at least 55° C., and preferably at least 60° C.;, and, except for during an evaporation/concentration step, typically in the range of from 45° Celsius to 90° Celsius (for example from 50° Celsius to 85° Celsius, preferably from 60° Celsius to 80° Celsius) after the end of fermentation and prior to the acidulation step. During an evaporation/concentration step where the lactic acid product concentration is being increased, the temperature typically is from 50° C. to 150° C., 60° C. to 140° C., 70° C. to 135° C., or 80° C. to 100° C., and in a particular preferred embodiment where higher vacuum is utilized, from 50° C. to 90° C., (preferably from 70° C. to 85° C., and more preferably 70° C. to 80° C.).

The calcium hydroxide-based pH control agent that comprises from about 0.1 to about 10 wt %, typically from 1.5 to 8.5 wt % silica to form a fermentation broth comprising lactic acid and calcium lactate. In an aspect, the calcium hydroxide-based pH control agent has a silica content of from about 2.0 to about 8.0% wt silica; or wherein the calcium hydroxide-based pH control agent has a silica content of from about 2.5 to 7.5 wt % silica; or wherein the calcium hydroxide-based pH control agent has a silica content of from about 3 to about 7 wt % silica.

In an aspect, the calcium hydroxide-based pH control agent used in the fermentation advantageously comprises less than 5 wt % silica. It has been found that calcium hydroxide-based pH control agents comprising greater than 5 wt % silica in the calcium hydroxide-based pH control agent, such as lime, may require additional adjustments of hydrocyclone parameters and/or profiling of the silica particle size distribution to provide efficient removal of the silica.

In an aspect, calcium hydroxide-based pH control agent is selected from calcium oxide, calcium hydroxide, or mixtures thereof. Due to its ready availability and cost, lime preferably is utilized.

After fermentation, the fermentation broth is directed from fermenter 102 to broth evaporator 105 to concentrate the broth.

The fermentation broth is heated to evaporate water from the broth, and advantageously (after evaporation to concentrate above 15 wt % lactic acid equivalents) the fermentation broth is maintained at a sufficiently high temperature (e.g., greater than about 50 degrees C. or greater than about 60 degrees C.) to prevent crystallization of calcium lactate.

If the fermentation broth is not evaporated to concentrate the lactic equivalents to at least 15 wt % prior to the centrifuge(s) being utilized, then the fermentation broth typically is maintained at a temperature of at least 30 degrees C., at least 35 degrees C., for example from 35-45 degrees C. or from 35-40 degrees C. during the centrifuge step if the concentration of lactic equivalents in the broth being process is less than 15 wt %. If the fermentation broth comprises at least 15 wt % lactic equivalents, then it typically is maintained at a temperature of at least 50° C., preferably at least 60° C.

In an aspect, the fermentation broth after concentration typically has a lactic equivalents concentration of from about 10 wt % to 30 wt % (for example from 15 wt % to 30 wt %). In an aspect, the fermentation broth after concentration has a concentration of from about 15 wt % to 25 wt % of lactic equivalents. In an aspect, the fermentation broth after concentration has a concentration of from about 1 8 wt % to 23 wt % of lactic equivalents. It has been found that low concentrations of lactic equivalent in the concentrated broth produced in this step result in excessive hydraulic loading to the overall system for recovering lactic acid, thereby reducing performance and/or efficiency and increasing energy usage. It has also been found that too high a concentration of lactic equivalents in the concentrated broth produced in step can result in an unacceptable amount of calcium lactate being insoluble.

In an aspect, the fermentation broth after concentration has a lactic equivalent concentration of at least about 150 g/L. In an aspect, the fermentation broth after concentration has a lactic equivalents concentration of at least about 200 g/L. In an aspect, the fermentation broth after concentration has a lactic equivalents concentration of at least about 250 g/L. In an aspect, the fermentation broth after concentration has a lactic equivalents concentration of at least about 300 g/L. In an aspect, the fermentation broth after concentration has a lactic equivalents concentration of from about 200 g/L to about 350 g/L. In an aspect, the fermentation broth after concentration has a lactic equivalents concentration of from about 200 g/L to about 330 g/L. In an aspect, the fermentation broth after concentration has a lactic equivalents concentration of from about 200 g/L to about 300 g/L. In an aspect, the fermentation broth after concentration has a lactic equivalents concentration of from about 250 g/L to about 350 g/L. In an aspect, the fermentation broth after concentration has a lactic equivalents concentration of from about 250 g/L to about 330 g/L. In an aspect, the fermentation broth after concentration has a lactic equivalents concentration of from about 250 g/L to about 300 g/L.

After concentration, the fermentation broth is directed from broth evaporator 105 to primary hydrocyclone 110 to separate silica from the lactic acid products and biomass.

In an aspect, the fermentation broth is directed to the primary hydrocyclone 110 at a temperature wherein at least 90 wt %, (preferably at least 95 wt %) of the calcium lactate present is solubilized. In an aspect, the fermentation broth is directed to the primary hydrocyclone 110 at a temperature wherein at least 97 wt % of the calcium lactate present is solubilized. In an aspect, the fermentation broth fermentation broth is directed to the primary hydrocyclone 110 at a temperature wherein at least 98 wt % of the calcium lactate present is solubilized. In an aspect, the fermentation broth fermentation broth is directed to the primary hydrocyclone 110 at a temperature wherein at least 99 wt % of the calcium lactate present is solubilized.

In an aspect, any of the streams of the processes described herein comprising calcium lactate are maintained at a temperature and calcium lactate concentration throughout the process such that at least 95% of the calcium lactate present is solubilized in the aqueous solution. In an aspect, any of the streams comprising calcium lactate are maintained at a temperature and calcium lactate concentration throughout the process such that at least 97% of the calcium lactate present is solubilized. In an aspect, any of the streams comprising calcium lactate are maintained at a temperature and calcium lactate concentration throughout the process such that at least 98% of the calcium lactate present is solubilized. In an aspect, any of the streams comprising calcium lactate are maintained at a temperature and calcium lactate concentration throughout the process such that at least 99% of the calcium lactate present is solubilized. For example, the pH typically is in the range of from 2.5 to 4.0 (for example from 2.7 to 3.5) after the end of fermentation and prior to the acidulation step described below; and, after the end of fermentation, the temperature typically is at least 45° C., at least 50° C., at least 55° C., and preferably at least 60° C.; and, except for during an evaporation/concentration step, typically in the range of from 45° Celsius to 90° Celsius (for example from 50° Celsius to 85° Celsius, preferably from 60° Celsius to 80° Celsius) after the end of fermentation and prior to the acidulation step. During an evaporation/concentration step where the lactic acid product concentration is being increased, the temperature typically is from 50° C. to 150° C., 60° C. to 140° C., 70° C. to 135° C., or 80° C. to 100° C., and in a particular preferred embodiment where higher vacuum is utilized, from 50° C. to 90° C., (preferably from 70° C. to 85° C., and more preferably 70° C. to 80° C.).

In an aspect, the fermentation broth is directed to the primary hydrocyclone 110 at a temperature of from about 60° C. to about 85° C. In an aspect, the fermentation broth fermentation broth is directed to the primary hydrocyclone 110 at a temperature of from about 70° C. to about 80° C.

In an aspect, any of the streams of the processes describe herein comprising calcium lactate are maintained at a temperature of from about 60° C. to about 85° C. In an aspect, any of the streams comprising calcium lactate are maintained at a temperature of from about 70° C. to about 80° C.

In an aspect, the fermentation broth as directed to the primary hydrocyclone 110 has a pH of from about 2.5 to about 5.0 (for example from 2.5 to 4.0). In an aspect, any of the streams of the processes described herein downstream of the fermenter and prior to the acidulation step that comprise calcium lactate are maintained at a pH of from about 2.7 to about 4.0, for example 2.8 to 3.5.

Primary hydrocyclone 110 separates the fermented product stream into a primary hydrocyclone overflow stream 111 comprising lactic acid and calcium lactate, biomass, water, wherein the primary hydrocyclone overflow stream contains no more than about 0.05 wt % of silica, and a primary hydrocyclone underflow stream 113 enriched in silica and comprising water, biomass, calcium lactate and lactic acid.

For purposes of the present discussion, a hydrocyclone (sometimes called a degritting hydrocyclone or a degritting cyclone) is a device to classify, separate or sort particles in a liquid suspension based on the ratio of their centripetal force to fluid resistance. This ratio is high for dense and coarse particles, and low for light and fine particles. The hydrocyclone is equipped with an inlet where the feed is provided, an upper outlet and a lower outlet, which is below the upper outlet. The upper outlet is located at or near the top of the hydrocyclone: the lower outlet is located at or near the bottom of the hydrocyclone. The hydrocyclone provides a preferential separation for solid product.

Degritting hydrocyclones operate under Stokes's Law (settling velocities) to remove silica from the incoming stream in preparation for centrifugation. These hydrocyclones separate the silica well due to the high density of the silica resulting in it settling at least 1Ox faster than biomass (depending on silica particle size). Hydrocyclones are commercially available, such as for example 3″ diameter×3.5′ long hydrocyclones manufactured by Fluid Quip.

Hydrocyclones used in the present process system, such as primary hydrocyclone 110 and secondary hydrocyclone 120, are installed to provide adequate flow (and thus pressure drop) across each hydrocyclone for effective silica removal.

In an aspect, the hydrocyclones used in the present process system, such as primary hydrocyclone 110 and secondary hydrocyclone 120, are provided as two or more hydrocyclones installed in series to provide adequate silica removal.

In an aspect, the hydrocyclones used in the present process system, such as primary hydrocyclone 110 and secondary hydrocyclone 120, are provided as two or more hydrocyclones installed in parallel to provide enhanced production rates using conveniently obtained hydrocyclone equipment.

In an aspect, the hydrocyclones used in the present process system, such as primary hydrocyclone 110 and secondary hydrocyclone 120, are operated under an initial pressure of at least about 30 psig.

In an aspect, the flow through the hydrocyclones used in the present process system, such as primary hydrocyclone 110 and secondary hydrocyclone 120, are regulated by valving cyclones on and off, and not by reducing feed pressure via a control valve.

In an aspect, the hydrocyclones used in the present process system, such as primary hydrocyclone 110 and secondary hydrocyclone 120, are provided with a strainer upstream of the hydrocyclone that is in turn configured for automatic periodic flush of the strainer, thereby preventing clogging of the strainer.

As noted above, the primary hydrocyclone overflow stream 111 comprises lactic acid and calcium lactate, biomass, water, and wherein the primary hydrocyclone overflow stream 111 comprises no more than about 0.05 wt % of silica. In an aspect, the primary hydrocyclone typically exhibits a flow split ratio wherein at least about 50% of the feed goes to the overflow stream. In an aspect, the primary hydrocyclone exhibits a flow split ratio wherein from about 70% to about 95% of the feed goes to the overflow stream. In an aspect, the primary hydrocyclone exhibits a flow split ratio wherein from about 85 to about 90% of the feed goes to the overflow stream.

As noted above, the primary hydrocyclone underflow stream 113 is enriched in silica and comprises water, biomass, lactic acid and calcium lactate.

Primary hydrocyclone underflow stream 113 is directed to secondary hydrocyclone 120 via optional secondary hydrocyclone feed tank 118. Secondary hydrocyclone 120 further separates silica present in the primary hydrocyclone underflow stream 113, and provides additional opportunity to recover lactic acid products that might be present in the primary hydrocyclone underflow stream 113. Optional secondary hydrocyclone feed tank 118 advantageously may provide better process control by decoupling the hydrocyclones, thereby preventing backpressure effects and allowing modification of the content and conditions of the material being fed to the secondary hydrocyclone 120.

Secondary hydrocyclone 120 thus conducts a separation of the primary hydrocyclone underflow stream 113 into secondary hydrocyclone overflow stream 121 that comprises lactic acid, calcium lactate, biomass, and water, wherein the secondary hydrocyclone 120 contains no more than 0.05 wt % of silica and secondary hydrocyclone underflow stream 123 that is enriched in silica and comprising water, biomass, calcium lactate and lactic acid.

In an aspect, the secondary hydrocyclone exhibits a flow split ratio wherein at least about 50% of the feed goes to the overflow stream. In an aspect, the secondary hydrocyclone exhibits a flow split ratio wherein from about 50% to about 85% of the feed goes to the overflow stream. In an aspect, the secondary hydrocyclone exhibits a flow split ratio wherein from about 65 to about 70% of the feed goes to the overflow stream.

Primary hydrocyclone overflow stream 111 and secondary hydrocyclone overflow stream 121 are directed to centrifuge 140 via optional centrifuge feed tank 138. Centrifuge 140 separates these streams into a clarified broth stream 141 comprising lactic acid, and calcium lactate, and a centrifuge concentrate stream 143 enriched in biomass.

In an aspect, the hydrocyclone array is configured so that about 90% or more of the lactic equivalents present in the fermentation broth stream 107 is fed to centrifuge 140 and greater than 92% of silica particles present in the fermentation broth stream 107 is directed to secondary hydrocyclone underflow stream 123. It has been found that reducing the amount of silica particles fed to centrifuge 140 substantially reduces centrifuge fouling, deterioration, and plugging.

The centrifuge separates the hydrocyclone overflow into a clarified broth stream comprising lactic acid products and a biomass heavy stream.

In an aspect, the centrifuge is a nozzle disc stack centrifuge that functions as the fine clarification step and produce the majority of the clarified broth.

In an aspect, the centrifuge has a heavy component ejection functionality to purge more dense material, including silica that may have been in the hydrocyclone overflow stream as a dense material purge separate from the biomass heavy stream. The ability to separately purge silica that may be present in the centrifuge is particularly advantageous in preventing silica build-up in the centrifuge vessel.

Clarified broth stream 141 is directed to acidulation station 160, where the clarified broth stream is acidulated with sulfuric acid to form aqueous lactic acid product (comprising substantially lactic acid (i.e. at least 90 wt % by total weight of the lactic acid product, preferably at least 95 wt %, more preferably at least 98 wt %, and most preferably at least 99 wt % lactic acid based on the total lactic acid product present), typically with less than ten (10) percent by weight of the lactic equivalents attributable to lactate salts, preferably less than five (5) percent by weight, for example, less than one (1) percent by weight of the lactic equivalents present attributable to lactate salts) and insoluble components comprising gypsum. The weight ratio of aqueous lactic acid product to gypsum typically ranging from 1:1 to 4:1 (for example 1.5:1 to 2.5:1). The aqueous lactic acid product is separated from the insoluble components comprising gypsum to provide aqueous lactic acid stream 171 and insoluble components stream 173. Separation may be carried out by any appropriate separation system (not shown), such as a decanter system, filtration system, or the like. Aqueous lactic acid stream 171 is directed to lactic acid collector 180 for subsequent handling, and optionally additional purification and concentration.

In an aspect, the aqueous lactic acid product is treated to provide an aqueous lactic acid product having a lactic acid concentration of at least 60 wt %. In an aspect, the aqueous lactic acid product is treated to provide an aqueous lactic acid product having a lactic acid concentration of at least 70 wt %. In an aspect, the aqueous lactic acid product is treated to provide an aqueous lactic acid product having a lactic acid concentration of at least 80 wt %. In an aspect, the aqueous lactic acid product is treated to provide an aqueous lactic acid product having a lactic acid concentration of at least 85 wt %. In an aspect, the aqueous lactic acid product is treated to provide an aqueous lactic acid product having a lactic acid concentration of at least 90 wt %. In an aspect, the aqueous lactic acid product is treated to provide an aqueous lactic acid product having a lactic acid concentration of at least 95 wt %.

In an aspect, the aqueous lactic acid product is treated by using a distillation process to increase the lactic acid concentration and remove remaining unwanted impurities. In an aspect, the distillation process comprises the use of a wiped film evaporator. In an aspect, the distillation process comprises the use of a distillation column. In an aspect, the distillation process comprises the use of a boiling tube evaporator or other type of evaporator known to one of skill in the art.

In an aspect, secondary hydrocyclone underflow stream 123, centrifuge concentrate stream 143 and insoluble components stream 173 are each directed to mixed biomass/gypsum collector 195 for subsequent handling. Alternatively, secondary hydrocyclone underflow stream 123 and centrifuge concentrate stream 143 may be directed to a biomass collector, and insoluble components stream 173 may be directed to a separate gypsum collector for subsequent handling. In an aspect, the biomass stream has a dry substance content of from about 30 to about 50 wt %.

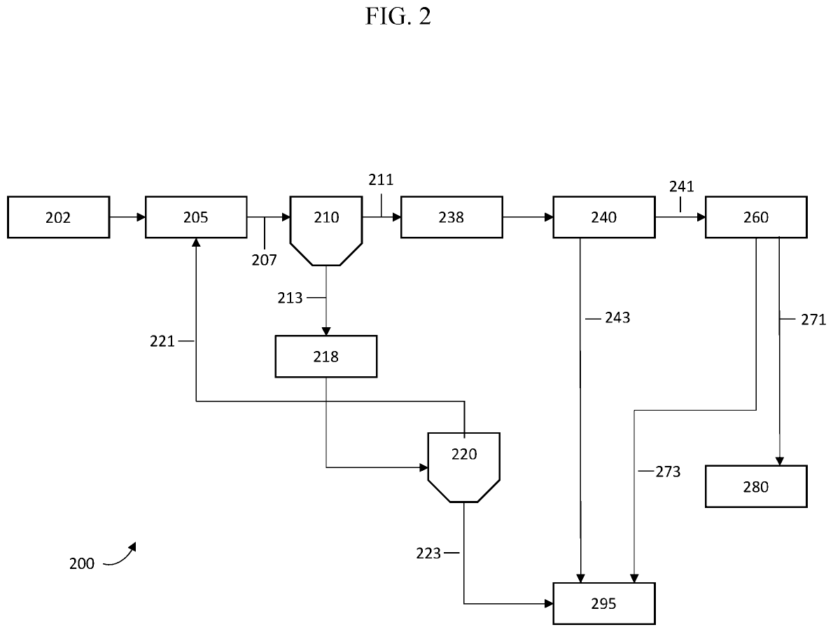

FIG. 2 is a schematic process flow diagram of an aspect of a process 200 for preparing lactic acid. In this process, a carbohydrate source is fermented as described above in fermenter 202.

After fermentation, the fermentation broth is directed from fermenter 202 to broth evaporator 205 to concentrate the broth. After concentration, the fermentation broth is directed from broth evaporator 205 to primary hydrocyclone 210 to separate silica from the biomass and lactic acid products as described above.

Primary hydrocyclone 210 separates the fermented product stream into a primary hydrocyclone overflow stream 211 comprising lactic acid, calcium lactate, biomass, water, and no more than about 0.05 wt % of silica, and a primary hydrocyclone underflow stream 213 enriched in silica and comprising water, biomass, calcium lactate, and lactic acid.

Primary hydrocyclone underflow stream 213 is directed to secondary hydrocyclone 220 via optional secondary hydrocyclone feed tank 218. Secondary hydrocyclone 220 further separates silica present in the primary hydrocyclone underflow stream 213, and provides additional opportunity to recover lactic acid products that might be present in the primary hydrocyclone underflow stream 213. Optional secondary hydrocyclone feed tank 218 advantageously may provide better process control by decoupling the hydrocyclone, thereby preventing backpressure effects and allowing modification of the content and conditions of the material being fed to the secondary hydrocyclone 220.

Secondary hydrocyclone 220 thus conducts a separation of the primary hydrocyclone underflow stream 213 into secondary hydrocyclone overflow stream 221 that comprises lactic acid, calcium lactate, biomass, water, and no more than 0.05 wt % of silica and secondary hydrocyclone underflow stream 223 that is enriched in silica and comprising water, biomass, lactic acid, and calcium lactate.

In contrast to the process as shown in FIG. 1, secondary hydrocyclone overflow stream 221 is directed to broth evaporator 205 for concentration and recycling into primary hydrocyclone 210 for further removal of silica that might be present in the secondary hydrocyclone overflow stream.

Primary hydrocyclone overflow stream 211 is directed to centrifuge 240 via optional centrifuge feed tank 238. Centrifuge 240 separates this stream into a clarified broth stream 241 comprising lactic acid and calcium lactate, and a centrifuge concentrate stream 243 enriched in biomass.

In an aspect, the hydrocyclone array is configured so that about 90% or more of the lactic acid products present in the fermentation broth stream 207 is fed to centrifuge 240 and greater than 92% of silica particles present in the fermentation broth stream 207 is directed to secondary hydrocyclone underflow stream 223.

Clarified broth stream 241 is directed to acidulation station 260, where the clarified broth stream is acidulated with sulfuric acid to form an aqueous lactic acid product (typically less than 5 percent by weight of the lactic equivalents in form of lactate anions, preferably less than 1 percent by weight of the lactic equivalents in the form of lactate anions) and insoluble components comprising gypsum. The aqueous lactic acid product is separated from the insoluble components comprising gypsum to provide aqueous lactic acid stream 271 and insoluble components stream 273. Separation may be carried out by any appropriate separation system (not shown), such as a decanter system, filtration system, or the like. Aqueous lactic acid stream 271 is directed to lactic acid collector 280 for subsequent handling.

In an aspect, secondary hydrocyclone underflow stream 223, centrifuge concentrate stream 243 and insoluble components stream 273 are each directed to mixed biomass/gypsum collector 295 for subsequent handling. Alternatively, secondary hydrocyclone underflow stream 223 and centrifuge concentrate stream 243 may be directed to a biomass collector, and insoluble components stream 273 may be directed to a separate gypsum collector for subsequent handling.

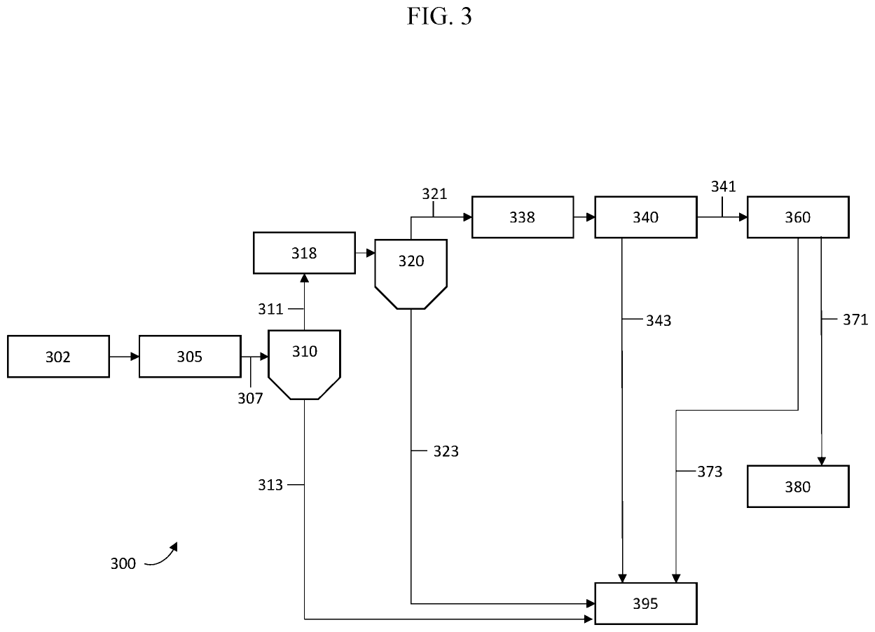

FIG. 3 is a schematic process flow diagram of an aspect of a process 300 for preparing lactic acid products. In this process, a carbohydrate source is fermented as described above in fermenter 302.

After fermentation, the fermentation broth is directed from fermenter 302 to broth evaporator 305 to concentrate the broth. After concentration, the fermentation broth is directed from broth evaporator 305 to primary hydrocyclone 310 to separate silica from the lactic acid products and biomass as described above.

Primary hydrocyclone 310 separates the fermented product stream into a primary hydrocyclone overflow stream 311 comprising lactic acid, calcium lactate, biomass, water, and no more than about 0.05 wt % of silica, and a primary hydrocyclone underflow stream 313 enriched in silica and comprising water, biomass, calcium lactate and lactic acid.

In contrast to the processes as shown in FIG. 1 and FIG. 2, primary hydrocyclone overflow stream 311 is directed to secondary hydrocyclone 320 via optional secondary hydrocyclone feed tank 318. Secondary hydrocyclone 320 further separates silica present in the primary hydrocyclone overflow stream 311. Optional secondary hydrocyclone feed tank 318 advantageously may provide better process control by decoupling the hydrocyclone, thereby preventing backpressure effects and allowing modification of the content and conditions of the material being fed to the secondary hydrocyclone 320.

Secondary hydrocyclone 320 thus conducts a separation of the primary hydrocyclone overflow stream 311 into secondary hydrocyclone overflow stream 321 that comprises lactic acid, calcium lactate, biomass, water, and no more than 0.05 wt % of silica and secondary hydrocyclone underflow stream 323 that is enriched in silica and comprising water, biomass, lactic acid and calcium lactate.

By directing primary hydrocyclone overflow stream 311 into secondary hydrocyclone 320 the stream material that is stream comprising lactic acid and calcium lactate is subjected to two sequential separation operations for further removal of silica that might be present in the stream.

Secondary hydrocyclone overflow stream 321 is directed to centrifuge 340 via optional centrifuge feed tank 338. Centrifuge 340 separates this stream into a clarified broth stream 341 comprising lactic acid and calcium lactate, and a centrifuge concentrate stream 343 enriched in biomass.

In an aspect, the hydrocyclone array is configured so that about 90% or more of the lactic acid product present in the fermentation broth stream 307 is fed to centrifuge 340 and greater than 92% of silica particles present in the fermentation broth stream 307 is directed to secondary hydrocyclone underflow stream 323.

Clarified broth stream 341 is directed to acidulation station 360, where the clarified broth stream is acidulated with sulfuric acid to form an aqueous lactic acid product (typically lactic equivalents present comprising less than five (5) percent by weight lactate anion, preferably less than one (10 percent by weight lactate anion) and insoluble components comprising gypsum. The aqueous lactic acid product is separated from the insoluble components comprising gypsum to provide aqueous lactic acid stream 371 and insoluble components stream 373. Separation may be carried out by any appropriate separation system (not shown), such as a decanter system, filtration system, or the like. Aqueous lactic acid stream 371 is directed to lactic acid collector 380 for subsequent handling.

In an aspect, primary hydrocyclone underflow stream 313, secondary hydrocyclone underflow stream 323, centrifuge concentrate stream 343 and insoluble components stream 373 are each directed to mixed biomass/gypsum collector 395 for subsequent handling. Alternatively, primary hydrocyclone underflow stream 313, secondary hydrocyclone underflow stream 323, and centrifuge concentrate stream 343 may be directed to a biomass collector, and insoluble components stream 373 may be directed to a separate gypsum collector for subsequent handling.

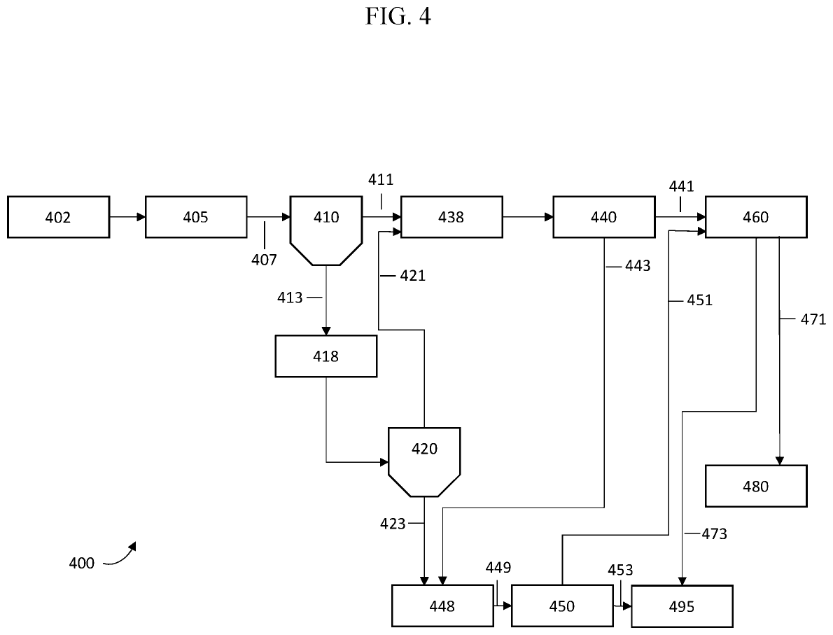

FIG. 4 is a schematic process flow diagram of an aspect of a process 400 for preparing lactic acid. In this process, a carbohydrate source is fermented as described above in fermenter 402.

After concentration, the fermentation broth is directed from broth evaporator 405 to primary hydrocyclone 410 to separate silica from the lactic acid products and biomass as described above.

Primary hydrocyclone 410 separates the fermented product stream into a primary hydrocyclone overflow stream 411 comprising lactic acid, calcium lactate, biomass, water, and no more than about 0.05 wt % of silica, and a primary hydrocyclone underflow stream 413 enriched in silica and comprising water, biomass, lactic acid and calcium lactate.

Primary hydrocyclone underflow stream 413 is directed to secondary hydrocyclone 420 via optional secondary hydrocyclone feed tank 418. Secondary hydrocyclone 420 further separates silica present in the primary hydrocyclone underflow stream 413, and provides additional opportunity to recovery lactic acid products that might be present in the primary hydrocyclone underflow stream 413. Optional secondary hydrocyclone feed tank 418 advantageously may provide better process control by decoupling the hydrocyclone, thereby preventing backpressure effects and allowing modification of the content and conditions of the material being fed to the secondary hydrocyclone 420.

Secondary hydrocyclone 420 thus conducts a separation of the primary hydrocyclone underflow stream 413 into secondary hydrocyclone overflow stream 421 that comprises lactic acid, calcium lactate, biomass, water, and no more than 0.05 wt % of silica and secondary hydrocyclone underflow stream 423 that is enriched in silica and comprising water, biomass, lactic acid and calcium lactate.

Primary hydrocyclone overflow stream 411 and secondary hydrocyclone overflow stream 421 are directed to centrifuge 440 via optional centrifuge feed tank 438. Centrifuge 440 separates these streams into a clarified broth stream 441 enriched in lactic acid products, and a centrifuge concentrate stream 443 enriched in biomass.

In an aspect, centrifuge 440 has a heavy component ejection functionality to purge more dense material, including silica that may have been in the hydrocyclone overflow stream as a dense material purge separate from the biomass heavy stream. The ability to separately purge silica that may be present in the centrifuge is particularly advantageous in preventing silica build-up in the centrifuge vessel. In an aspect, the dense material purge is directed to decanter/centrifuge 450 as discussed below.

Secondary hydrocyclone underflow stream 423 and centrifuge concentrate stream 443 are directed to decanter/centrifuge 450 via optional decanter/centrifuge feed collector 448. Decanter/centrifuge 450 separates these streams into centrate 451 comprising lactic acid products, and decanter/centrifuge underflow 453 enriched in silica and biomass. Optional decanter/centrifuge biomass feed collector 448 advantageously may provide better process control by decoupling decanter/centrifuge 450 from the hydrocyclones, thereby preventing backpressure effects and allowing modification of the content and conditions of the material being fed to decanter/centrifuge 450. Alternatively, the secondary hydrocyclone underflow stream 423 and the centrifuge concentrate stream 443 are combined together by metering each stream directly into the decanter/centrifuge as a decanter/centrifuge biomass feed.

For purposes of the present discussion, a decanter/centrifuge is a device used for separation of a feed slurry into predominantly solid phase (that may contain aqueous components) and a liquid stream. The decanter/centrifuge comprises a feed port (where the slurry comprising lactic acid, calcium lactate, water, silica, and some biomass (typically at least 1.5 percent by weight, and preferably at least 2 wt % (for example, from 5 to 20 percent by weight) is introduced), a decanter bowl, solid phase discharge outlet, and a liquid phase outlet. In operation, the decanter bowl rotates and the solids in the feed slurry settle on the inner wall of the decanter bowl. A conveyor continuously removes the solids from the decanter bowl through the solid phase discharge outlet. The decanter bowl can be configured horizontally or vertically. See, e.g. zkcentrifuge.com/product/decanter.html on the World Wide Web; EP0824379B1; U.S. Pat. No. 7,255,670 and flottweg.com/product-lines/sedicanterr on the World Wide Web, the disclosures of which are incorporated herein by reference.

For the current method, the solids discharged from the decanter/centrifuge comprises primarily biomass with some silica, and other solids present in the slurry. The liquid stream discharged through liquid phase outlet comprises aqueous lactic acid and aqueous calcium lactate.

Preferably, the decanter/centrifuge is operated with the decanter bowl configured in a substantially horizontal arrangement (e.g., with the longitudinal axis of the bowl preferably at a less than a 5% angle with respect to horizontal).

In an aspect, the decanter/centrifuge comprises a bowl configured with a side ejection port to purge the decanter/centrifuge underflow stream 453 from the bowl. In an aspect, the decanter/centrifuge comprises a set of interchangeable bowls such that one bowl can be removed and rapidly exchanged with another bowl.

In an aspect, the materials fed to the decanter/centrifuge should have a solids content of at least about 1.5 wt % solids (preferably at least 2.0 wt % (for example from 5 wt % to 20 wt %) for efficient operation. When the solids content of the decanter/centrifuge feed is too low, the cake may wash out of the decanter/centrifuge through the biomass discharge. If the cake is allowed to wash out, it must be rebuilt up over time, with loss of productivity and potential loss of final lactic acid product.

In an aspect, it has been found that it is advantageous to add water to the decanter/centrifuge feed stream 449 prior to directing the feed to the decanter/centrifuge 450. Addition of water at this stage of the process has been found to assure that calcium lactate that may be present in the feed is solubilized, and therefore will be recovered in the centrate 451 to contribute to the overall yield of lactic acid.

In an aspect, the decanter/centrifuge feed stream 449 has a dry substance content of from about 2 to about 20%, or wherein the decanter/centrifuge feed stream 449 has a dry substance content of from about 4 to about 15%, or wherein the decanter/centrifuge feed steam 449 has a dry substance content of from about 5 to about 10%.

Typically, the flow of feed streams to the decanter/centrifuge 450 is controlled such that the decanter/centrifuge feed rate is operated at no less than half of maximum operational rate of the feed input to the centrifuge, thereby maintaining an acceptable high concentration of the centrifuge concentrate stream 443 that in turn maintains an appropriate decanter/centrifuge feed stream dry substance content for operation of the decanter/centrifuge.

In an aspect, centrate 451 typically comprises less than 0.2 wt % biomass. Centrate 451 typically is sent to the acidulation station or optionally to the inlet of the centrifuge 440. Preferably centrate 451 is sent to the inlet of centrifuge 440 when it comprises more than one weight percent (1 wt %) biomass.

In an aspect, decanter/centrifuge underflow 453 has a dry substance content of at least 30%, or at least 35%, or at least 40%. In an aspect, decanter/centrifuge underflow 453 has a viscosity of greater than 5000 Pa s.

In an aspect, the hydrocyclone array is configured so that about 90% or more of the lactic equivalents present in the fermentation broth stream 407 is fed to centrifuge 440 and greater than 92% of silica particles present in the fermentation broth stream 407 is directed to decanter/centrifuge 450. It has been found that reducing the amount of silica particles fed to centrifuge 440 substantially reduces centrifuge fouling and plugging.

Clarified broth stream 441 and optionally centrate 451 are directed to acidulation station 460, where the clarified broth stream is acidulated with sulfuric acid to form an aqueous lactic acid product (typically, less than five percent by weight (5 wt %) of the lactic equivalents present in the form of lactate anions, preferably less than one percent by weight (1 wt % of the lactic equivalents present in the form of lactate anions) and insoluble components comprising gypsum. The aqueous lactic acid product is separated from the insoluble components comprising gypsum to provide aqueous lactic acid stream 471 and insoluble components stream 473 (comprising gypsum). Separation of the insoluble components may be carried out by any appropriate separation system (not shown), such as a decanter system, filtration system(s) such as belt and/or drum filters), and/or the like. Aqueous lactic acid stream 471 is directed to lactic acid collector 480 for subsequent handling and further purification and/or concentration if desired.

Insoluble components stream 473 and decanter/centrifuge underflow 453 are each directed to mixed biomass/gypsum collector 495 for subsequent handling. Alternatively, decanter/centrifuge underflow 453 may be directed to a biomass collector, and insoluble components stream 473 may be directed to a separate gypsum collector for subsequent handling.

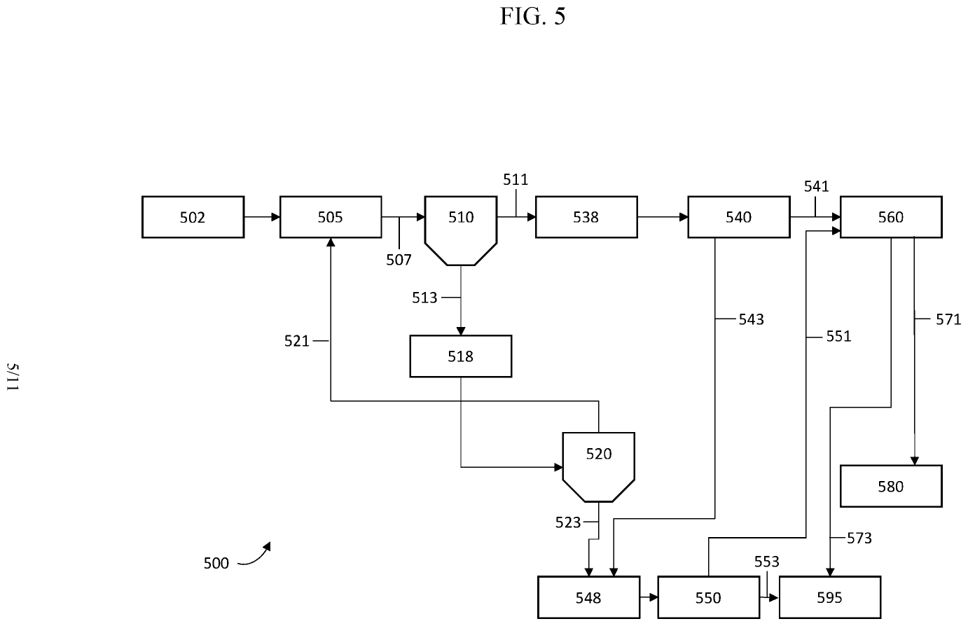

FIG. 5 is a schematic process flow diagram of an aspect of a process 500 for preparing lactic acid. In this process, a carbohydrate source is fermented as described above in fermenter 502.

After concentration, the fermentation broth is directed from broth evaporator 505 to primary hydrocyclone 510 to separate silica from the lactic acid products and biomass as described above.

Primary hydrocyclone 510 separates the fermented product stream into a primary hydrocyclone overflow stream 511 comprising lactic acid, calcium lactate, biomass, water, and no more than about 0.05 wt % of silica, and a primary hydrocyclone underflow stream 513 enriched in silica and comprising water, biomass, calcium lactate and lactic acid.

Primary hydrocyclone underflow stream 513 is directed to secondary hydrocyclone 520 via optional secondary hydrocyclone feed tank 518. Secondary hydrocyclone 520 further separates silica present in the primary hydrocyclone underflow stream 513, and provides additional opportunity to recover lactic acid products that might be present in the primary hydrocyclone underflow stream 513. Optional secondary hydrocyclone feed tank 518 advantageously may provide better process control by decoupling the hydrocyclone, thereby preventing backpressure effects and allowing modification of the content and conditions of the material being fed to the secondary hydrocyclone 520.

Secondary hydrocyclone 520 thus conducts a separation of the primary hydrocyclone underflow stream 513 into secondary hydrocyclone overflow stream 521 that comprises lactic acid, calcium lactate, biomass, water, and no more than 0.05 wt % of silica and secondary hydrocyclone underflow stream 523 that is enriched in silica and comprising water, biomass, lactic acid and calcium lactate.

Secondary hydrocyclone overflow stream 521 is directed to broth evaporator 505 for concentration and recycling into primary hydrocyclone 510 for further removal of silica that might be present in the secondary hydrocyclone overflow stream.

Primary hydrocyclone overflow stream 511 is directed to centrifuge 540 via optional centrifuge feed tank 538. Centrifuge 540 separates this stream into a clarified broth stream 541 comprising lactic acid and calcium lactate, and a centrifuge concentrate stream 543 enriched in biomass.

In an aspect, centrifuge 540 has a heavy component ejection functionality to purge more dense material, including silica that may have been in the hydrocyclone overflow stream as a dense material purge separate from the biomass heavy stream. The ability to separately purge silica that may be present in the centrifuge is particularly advantageous in preventing silica build-up in the centrifuge vessel. In an aspect, the dense material purge is directed to decanter/centrifuge 550 as discussed below.

Secondary hydrocyclone underflow stream 523 and centrifuge concentrate stream 543 are directed to decanter/centrifuge 550 via optional decanter/centrifuge biomass feed collector 548. Decanter/centrifuge 550 separates these streams into centrate 551 comprising in lactic acid and calcium lactate, and decanter/centrifuge underflow 553 enriched in silica and biomass. Optional decanter/centrifuge feed collector 548 advantageously may provide better process control by decoupling decanter/centrifuge 550 from the hydrocyclones, thereby preventing backpressure effects and allowing modification of the content and conditions of the material being fed to decanter/centrifuge 550. Alternatively, the secondary hydrocyclone underflow stream 523 and the centrifuge concentrate stream 543 are combined together by metering each stream directly into the decanter/centrifuge as a decanter/centrifuge biomass feed.

In an aspect, centrate 551 comprises less than 0.2 wt % biomass. Centrate 551 typically is sent to the acidulation station or optionally to the inlet of the centrifuge 540. Preferably Centrate 551 is sent to the inlet of centrifuge 540 when it comprises more than one weight percent (1 wt %) biomass.

In an aspect, decanter/centrifuge underflow 553 has a dry substance content of at least 30%, or at least 35%, or at least 40%. In an aspect, decanter/centrifuge underflow 553 has a viscosity of greater than 5000 Pa s.

In an aspect, the hydrocyclone array is configured so that about 90% or more of the lactic equivalents present in the fermentation broth stream 507 is fed to centrifuge 540 and greater than 92% of silica particles present in the fermentation broth stream 507 is directed to secondary hydrocyclone underflow stream 523.

Clarified broth stream 541 and optionally centrate 551 are directed to acidulation station 560, where the clarified broth stream is acidulated with sulfuric acid to form an aqueous lactic acid product (typically the lactic equivalents present comprising less than five percent by weight (5 wt %) lactate anions, preferably less than one percent by weight (1 wt %) lactate anions) and insoluble components comprising gypsum. The aqueous lactic acid product is separated from the insoluble components comprising gypsum to provide aqueous lactic acid stream 571 and insoluble components stream 573. Separation may be carried out by any appropriate separation system (not shown), such as a decanter system, filtration system (e.g., drum and belt filters), and/or the like. Aqueous lactic acid stream 571 is directed to lactic acid collector 580 for subsequent handling and further purification and/or concentration if desired.

Insoluble components stream 573 and decanter/centrifuge underflow 553 are each directed to mixed biomass/gypsum collector 595 for subsequent handling. Alternatively, decanter/centrifuge underflow 553 may be directed to a biomass collector, and insoluble components stream 573 may be directed to a separate gypsum collector for subsequent handling.

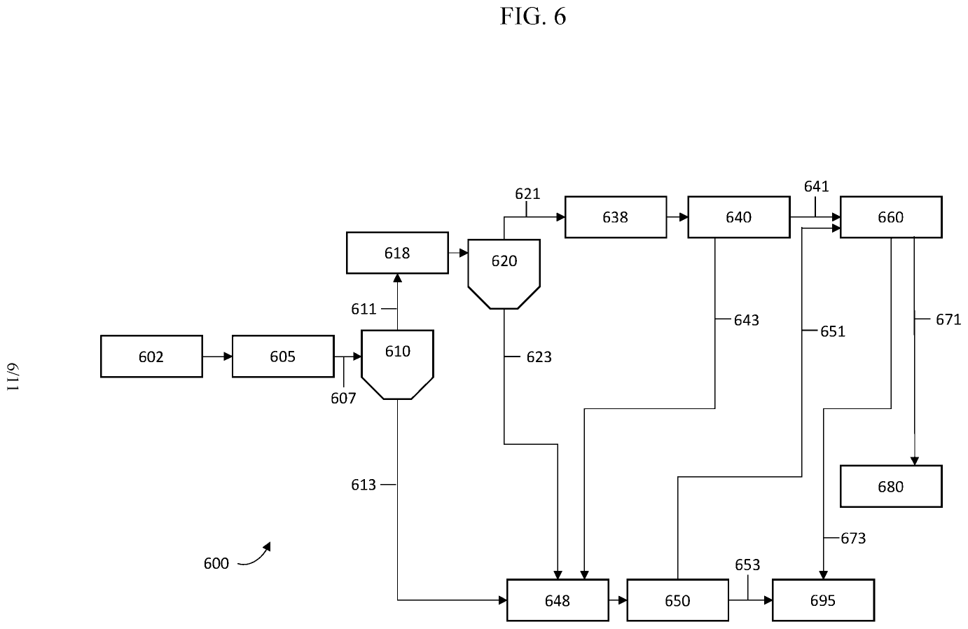

FIG. 6 is a schematic process flow diagram of an aspect of a process 600 for preparing lactic acid. In this process, a carbohydrate source is fermented as described above in fermenter 602.

After concentration, the fermentation broth is directed from broth evaporator 605 to primary hydrocyclone 610 to separate silica from the lactic acid products and biomass as described above.

Primary hydrocyclone 610 separates the fermented product stream into a primary hydrocyclone overflow stream 611 comprising lactic acid, calcium lactate, biomass, water, and no more than about 0.05 wt % of silica, and a primary hydrocyclone underflow stream 613 enriched in silica and comprising water, biomass, calcium lactate and lactic acid.

In contrast to the processes as shown in FIG. 4 and FIG. 5, primary hydrocyclone overflow stream 611 is directed to secondary hydrocyclone 620 via optional secondary hydrocyclone feed tank 618. Secondary hydrocyclone 620 further separates silica present in the primary hydrocyclone overflow stream 611. Optional secondary hydrocyclone feed tank 618 advantageously may provide better process control by decoupling the hydrocyclone, thereby preventing backpressure effects and allowing modification of the content and conditions of the material being fed to the secondary hydrocyclone 620.

Secondary hydrocyclone 620 thus conducts a separation of the primary hydrocyclone overflow stream 611 into secondary hydrocyclone overflow stream 621 that comprises lactic acid, calcium lactate biomass, water, and no more than 0.05 wt % of silica and secondary hydrocyclone underflow stream 623 that is enriched in silica and comprising water, lactic acid, and calcium lactate.

By directing primary hydrocyclone overflow stream 611 into secondary hydrocyclone 620 the stream material that comprises lactic acid and calcium lactate is subjected to two sequential separation operations for further removal of silica that might be present in the stream.

Secondary hydrocyclone overflow stream 621 is directed to centrifuge 640 via optional centrifuge feed tank 638. Centrifuge 640 separates this stream into a clarified broth stream 641 comprising lactic acid and calcium lactate, and a centrifuge concentrate stream 643 enriched in biomass.

In an aspect, centrifuge 640 has a heavy component ejection functionality to purge more dense material, including silica that may have been in the hydrocyclone overflow stream as a dense material purge separate from the biomass heavy stream. The ability to separately purge silica that may be present in the centrifuge is particularly advantageous in preventing silica build-up in the centrifuge vessel. In an aspect, the dense material purge is directed to decanter/centrifuge 650 as discussed below.

Primary hydrocyclone underflow stream 613, secondary hydrocyclone underflow stream 623 and centrifuge concentrate stream 643 are directed to decanter/centrifuge 650 via optional decanter/centrifuge feed collector 648. Decanter/centrifuge 650 separates these streams into centrate 651 enriched in lactic acid and calcium lactate, and decanter/centrifuge underflow 653 enriched in silica and biomass. Optional decanter/centrifuge biomass feed collector 648 advantageously may provide better process control by decoupling decanter/centrifuge 650 from the hydrocyclones, thereby preventing backpressure effects and allowing modification of the content and conditions of the material being fed to decanter/centrifuge 650. Alternatively, the primary hydrocyclone underflow stream 613, secondary hydrocyclone underflow stream 623 and the centrifuge concentrate stream 643 are combined together by metering each stream directly into the decanter/centrifuge as a decanter/centrifuge biomass feed.

In an aspect, centrate 651 comprises less than 0.2 wt % biomass. Centrate 651 typically is sent to the acidulation station or optionally to the inlet of the centrifuge 640. Preferably Centrate 651 is sent to the inlet of centrifuge 640 when it comprises more than one weight percent (1 wt %) biomass.

In an aspect, decanter/centrifuge underflow 653 has a dry substance content of at least 30%, or at least 35%, or at least 40%. In an aspect, decanter/centrifuge underflow 653 has a viscosity of greater than 5000 Pa s.

Clarified broth stream 641 and centrate 651 are directed to acidulation station 660, where the clarified broth stream is acidulated with sulfuric acid to form an aqueous lactic acid product (typically the lactic acid equivalents comprising less than five percent by weight (5 wt %), preferably less than one percent by weight (1 wt %) lactate anions) and insoluble components comprising gypsum. The aqueous lactic acid product is separated from the insoluble components comprising gypsum to provide aqueous lactic acid stream 671 and insoluble components stream 673. Separation may be carried out by any appropriate separation system (not shown), such as a decanter system, filtration system, or the like. Aqueous lactic acid stream 671 is directed to lactic acid collector 680 for subsequent handling.

Insoluble components stream 673 and decanter/centrifuge underflow 653 are each directed to mixed biomass/gypsum collector 695 for subsequent handling. Alternatively, decanter/centrifuge underflow 653 may be directed to a biomass collector, and insoluble components stream 673 may be directed to a separate gypsum collector for subsequent handling.

In an aspect, the hydrocyclone array is configured so that about 90% or more of the lactic equivalents present in the fermentation broth stream 607 is fed to acidulation station 660, and greater than 92% of silica particles present in the fermentation broth stream 607 is directed to decanter/centrifuge underflow stream 653.

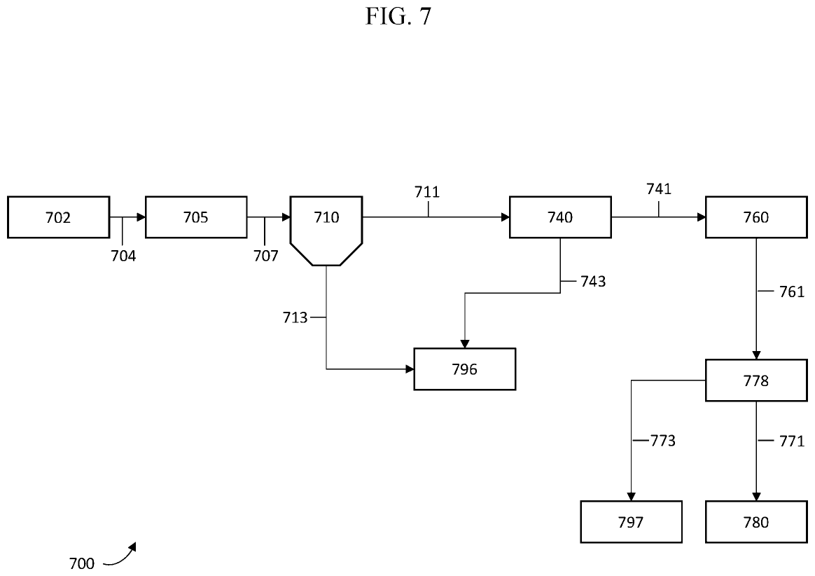

FIG. 7 is a schematic process flow diagram of an aspect of a process 700 for preparing lactic acid. In this process, a carbohydrate source is fermented as described above in fermenter 702.

After fermentation, the fermentation broth stream 704 is directed from fermenter 702 to broth evaporator 705 to concentrate the broth. After concentration, the evaporated fermentation broth stream 707 is directed from broth evaporator 705 to hydrocyclone 710 to separate silica from the lactic acid products and biomass.

Hydrocyclone 710 separates the fermented product stream into a hydrocyclone overflow stream 711 comprising lactic acid, calcium lactate, biomass, water, and no more than about 0.05 wt % of silica, and a hydrocyclone underflow stream 713 enriched in silica and comprising water, biomass, lactic acid and calcium lactate.

Hydrocyclone overflow stream 711 is directed to centrifuge 740 via optional centrifuge feed tank (not shown). Centrifuge 740 separates these streams into a clarified broth stream 741 comprising lactic acid and calcium lactate, and a centrifuge concentrate stream 743 enriched in biomass.

Clarified broth stream 741 is directed to acidulation station 760, where the clarified broth stream is acidulated with sulfuric acid to form an aqueous lactic acid product (typically the lactic acid equivalents comprising less than five percent by weight (5 wt %), preferably less than one percent by weight (1 wt %) lactate anions) and insoluble components comprising gypsum. The materials thus produced in acidulation station 760 are directed as aqueous lactic acid/gypsum stream 761 to lactic acid and gypsum separator 778. Lactic acid and gypsum separator 778 may be any appropriate separation system, such as a decanter system, filtration system (e.g., belt and/or drum filters), and/or the like. Aqueous lactic acid product is separated from the insoluble components comprising gypsum to provide aqueous lactic acid stream 771 and gypsum stream 773. Aqueous lactic acid stream 771 is directed to lactic acid collector 780 for subsequent handling (and if desired further purification and/or concentration), and gypsum stream 773 is directed to gypsum collector 797 for subsequent handling.

Centrifuge concentrate stream 743 and hydrocyclone underflow stream 713 are directed to biomass collector 796 for subsequent handling. Alternatively, hydrocyclone underflow stream 713, which is enriched in silica, may be collected for handling separately from the biomass.

Process 700 thus provides a simple process where silica present in the calcium hydroxide-based pH control agent is removed early in the process to avoid damage to filtration or centrifugation equipment, and where the aqueous lactic acid primary desired product and additional desirable co-products of biomass and gypsum can be collected in compositions and formats favorable to subsequent economic use.

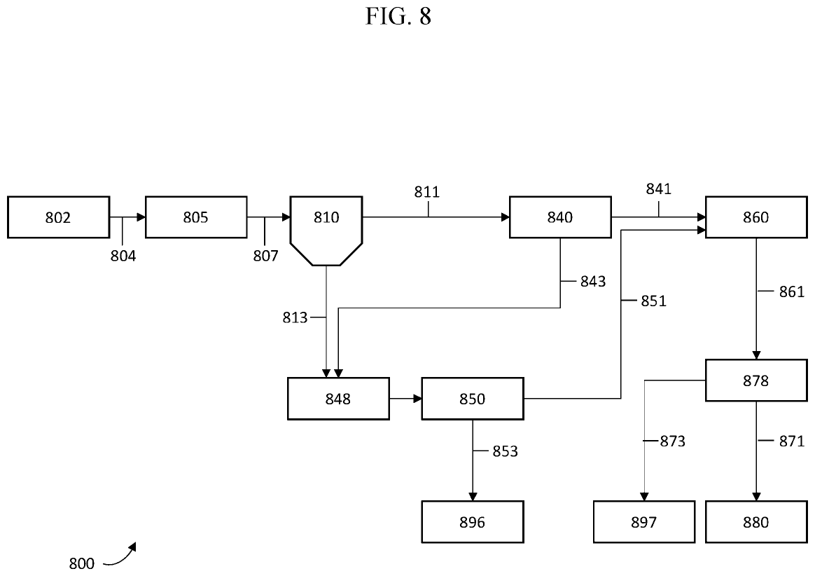

FIG. 8 is a schematic process flow diagram of an aspect of a process 800 for preparing lactic acid. In this process, a carbohydrate source is fermented as described above in fermenter 802.

After fermentation, the fermentation broth stream 804 is directed from fermenter 802 to broth evaporator 805 to concentrate the broth. After concentration, the evaporated fermentation broth stream 807 is directed from broth evaporator 805 to hydrocyclone 810 to separate silica from the lactic acid product (lactic acid and calcium lactate) and biomass.

Hydrocyclone 810 separates the fermented product stream into a hydrocyclone overflow stream 811 comprising lactic acid, calcium lactate, biomass, water, and no more than about 0.05 wt % of silica, and a hydrocyclone underflow stream 813 enriched in silica and comprising water, biomass, lactic acid and calcium lactate.

Hydrocyclone overflow stream 811 is directed to centrifuge 840 via optional centrifuge feed tank (not shown). Centrifuge 840 separates these streams into a clarified broth stream 841 comprising lactic acid and calcium lactate, and a centrifuge concentrate stream 843 enriched in biomass.

Hydrocyclone underflow stream 813 and centrifuge concentrate stream 843 are directed to decanter/centrifuge 850 via optional decanter/centrifuge feed collector 848. Decanter/centrifuge 850 separates these streams into centrate 851 enriched in lactic acid products (lactic acid and calcium lactate), and decanter/centrifuge underflow 853 enriched in silica and biomass. Optional decanter/centrifuge biomass feed collector 848 advantageously may provide better process control by decoupling decanter/centrifuge 850 from the hydrocyclone, thereby preventing backpressure effects and allowing modification of the content and conditions of the material being fed to decanter/centrifuge 850. Alternatively, the hydrocyclone underflow stream 813 and the centrifuge concentrate stream 843 are combined together by metering each stream directly into the decanter/centrifuge as a decanter/centrifuge biomass feed.

In an aspect, centrate 851 comprises less than 0.2 wt % biomass. Centrate 851 typically is sent to the acidulation station or optionally to the inlet of the centrifuge 840. Preferably Centrate 851 is sent to the inlet of centrifuge 840 when it comprises more than one weight percent (1 wt %) biomass.

Clarified broth stream 841 and optionally centrate 851 are directed to acidulation station 860, where the clarified broth stream is acidulated with sulfuric acid to form an aqueous lactic acid product (typically the lactic acid equivalents comprising less than five percent by weight (5 wt %), preferably less than one percent by weight (1 wt %) lactate anions) and insoluble components comprising gypsum. The materials thus produced in acidulation station 860 are directed as aqueous lactic acid/gypsum stream 861 to lactic acid and gypsum separator 878. Lactic acid and gypsum separator 878 may be any appropriate separation system, such as a decanter system, filtration system (for example, drum and belt filters), and/or the like. Aqueous lactic acid product is separated from the insoluble components comprising gypsum to provide aqueous lactic acid stream 871 and gypsum stream 873. Aqueous lactic acid stream 871 is directed to lactic acid collector 880 for subsequent handling (and if desired further purification and/or concentration), and gypsum stream 873 is directed to gypsum collector 897 for subsequent handling. Decanter/centrifuge underflow 853 is directed to biomass collector 896 for subsequent handling.

In an aspect, the present process provides the ability to make fermentation products on a production scale level with excellent yields and acceptably low impurity content. In an embodiment, systems using the present process are configured to produce batches of at least 25,000 gallons aqueous lactic acid product.

In an aspect, the biomass component obtained in the present process is useful as a feed product, such as a cattle feed. In an aspect, the biomass component obtained in the present process is useful, with or without addition of gypsum, for use as mulch.

In an aspect, the biomass component obtained in the present process is useful in production of energy such as electricity, in production of biofuels, and in production of biogas.

All percentages and ratios used herein are weight percentages and ratios unless otherwise indicated. All patents, patent applications (including provisional applications), and publications cited herein are incorporated by reference as if individually incorporated for all purposes. Numerous characteristics and advantages of the invention meant to be described by this document have been set forth in the foregoing description. It is to be understood, however, that while particular forms or embodiments of the invention have been illustrated, various modifications, including modifications to shape, and arrangement of parts, and the like, can be made without departing from the spirit and scope of the invention.

### EXAMPLES

Representative embodiments of the present disclosure will now be described with reference to the following example that illustrates the principles and practice of the present invention.

### Example 1

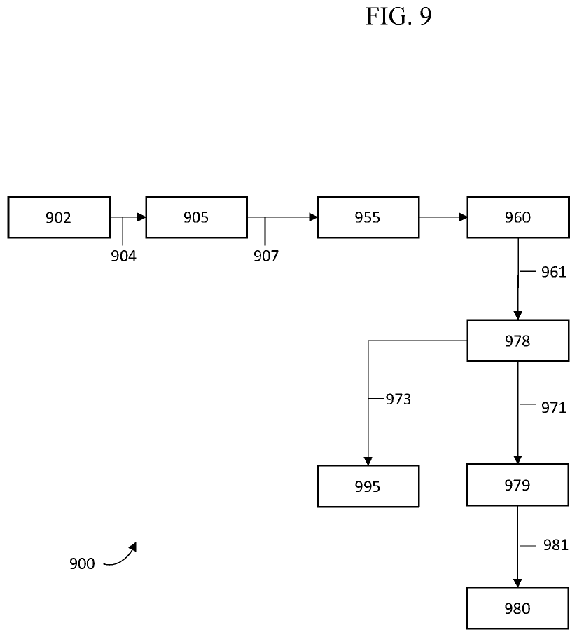

This example demonstrates the compositions of various process intermediate present in the process outlined in FIG. 9. The process in FIG. 9 and this example are for comparative purposes to demonstrate the flow of silica and biomass through a lactic acid preparation process that does not include and of the hydrocyclone or centrifuge unit operations that are part of the present invention.

In this example, soluble silica and calcium were measured using an inductively coupled plasma optical emission spectrometer (sold commercially as SPECTRO ARCOS ICP-OES analyzer by SPECTRO Analytical Instruments). As outlined herein, lactic acid fermentation utilizes calcium-hydroxide based pH control agents that contain silica. Accordingly, both calcium (residual from the pH control agents such as lime) and silica (introduced through the pH control agent) are measured to track their presence and abundance through the processes described herein.

Silica volume percentage (% vol) was measured by centrifugation of a sample (spun at 2700 g-force for 5 minutes, on the Hettich Universal 320 centrifuge instrument used this was 4500 rpm), and visual inspection of the volume of silica present relative to the total volume of the sample.

Nitrogen was measured using a nitrogen and protein analyzer with a thermal conductivity detector (sold commercially as Rapid MAX N Exceed by Elementar) that measures nitrogen content through sample combustion. The nitrogen content of a given sample is used as a proxy for the amount of biomass in the system. Total nitrogen in a sample is measured as well as a nitrogen content for an equivalent sample that has been filtered to remove solids. The difference between the total nitrogen and filtered nitrogen content is the amount of nitrogen contributed by any biomass present. When the filtered nitrogen content is higher than the total nitrogen content, all biomass has been removed and the filtered nitrogen content is higher because it has been enriched by the filtration process.

As outline in FIG. 9, the process 900 begins with production of the fermentation broth 904 in the fermenter 902, which is then to broth evaporator 905 to concentrate the broth and produce an evaporated fermentation broth stream 907. The evaporated fermentation broth stream is delivered to the acidification station 960, optionally though an acidification storage tank 955. Following acidification, the aqueous lactic acid/gypsum stream 961 is delivered to a lactic acid/gypsum separator 978 to separate an aqueous lactic acid stream 971 from an insoluble components stream 973 containing biomass, gypsum, and silica. The insoluble components stream 973 is directed to a mixed biomass/gypsum collector 995 and the aqueous lactic acid stream 971 is passed through a filter 979 after which the filtered aqueous lactic acid stream 981 is delivered to the lactic acid collector 980.

Three different fermentation broth samples were run through the process outlined in FIG. 9 and samples were taken from the fermentation broth stream 904, evaporated fermentation broth stream 907, aqueous lactic acid/gypsum stream 961, the aqueous lactic acid stream 971, and the filtered aqueous lactic acid stream 981. Table 2 reports the calcium, soluble silica, silica volume percent (0% vol), nitrogen, filtered nitrogen, and nitrogen difference for each of the samples.

The lactic acid fermentation broths tested in this example are outlined in Table 1. These broths contained lactic acid in addition to other fermentation by-products and fermentation components, including calcium (from lime pH control agent), silica (from line pH control agent), and nitrogen (proxy to measure biomass). These fermentation broths are the output of fermenter 902 in FIG. 9.

TABLE 1

| | Fermentation | Fermentation | Fermentation |
|---|---|---|---|
| Component | Broth A | Broth B | Broth C |
| Lactic acid (g/L) | 182.9 | 166.9 | 162.1 |
| Calcium (ppm) | 13099 | 13018 | 11638 |
| Soluble silica (ppm) | 66.62 | 66.46 | 61.28 |
| Silica (% vol) | 0.667 | 0.667 | 0.667 |
| Nitrogen (ppm) | 429.3 | 451.0 | 423.6 |
| Filtered nitrogen | 377.0 | 423.6 | 332.6 |
| (ppm) | | | |
| Nitrogen Difference | 52.4 | 27.5 | 151.2 |
| (ppm) | | | |
| Lactic acid (g/L) | 298.6 | 265.6 | 257.8 |
| after evaporation | | | |

TABLE 2

| | Broth Component | 904 | 907 | 961 | 971 | 981 |
|---|---|---|---|---|---|---|
| Fermentation | Calcium (ppm) | 13099 | 19724 | 2733 | 733.8 | 692.7 |
| Broth | Soluble Silica (ppm) | 66.62 | 112.31 | 109.21 | 99.49 | 96.24 |
| A | Silica (% vol) | 0.0667 | 0.1333 | 0.1330 | 0.1333 | 0.0000 |
| | Nitrogen (ppm) | 429.3 | 808.5 | 712.4 | 568.7 | 410.3 |
| | Filtered nitrogen (ppm) | 377.0 | 616.6 | 597.7 | 544.7 | 459.8 |
| | Nitrogen Difference (ppm) | 52.4 | 191.9 | 114.7 | 24.0 | −49.5 |
| Fermentat [symbol] | Calcium (ppm) | 13018 | 18529 | 2184 | 713.1 | 657.8 |
| | Soluble Silica (ppm) | 66.46 | 94.78 | 96.97 | 88.05 | 80.78 |
| | Silica (% vol) | 0.0667 | 0.1333 | 0.1330 | 0.1000 | 0.0000 |
| | Nitrogen (ppm) | 451.0 | 626.3 | 603.0 | 450.6 | 304.1 |
| | Filtered nitrogen (ppm) | 423.6 | 442.2 | 498.9 | 486.0 | 388.8 |
| | Nitrogen Difference (ppm) | 27.5 | 184.1 | 104.1 | −35.4 | −84.7 |
| Fermentation | Calcium (ppm) | 11638 | 16548 | 3468 | 728.2 | 738.8 |
| Broth | Soluble Silica (ppm) | 61.28 | 88.15 | 90.28 | 84.35 | 93.74 |
| C | Silica (% vol) | 0.0667 | 0.1333 | 0.1330 | 0.1333 | 0.0000 |
| | Nitrogen (ppm) | 483.8 | 691.7 | 636.8 | 473.9 | 338.8 |
| | Filtered nitrogen (ppm) | 332.6 | 369.0 | 466.2 | 510.1 | 391.3 |
| | Nitrogen Difference (ppm) | 151.2 | 322.7 | 170.6 | −36.2 | −52.5 |

[symbol]

indicates data missing or illegible when filed

The results show that after the stream is passed through both the lactic acid/gypsum separator 979 and the filter, all of the silica and the biomass has been removed. For all three fermentation broths testing in the process of FIG. 9, the nitrogen different in the filtered aqueous lactic acid stream 981 was negative, indicating that all of the solid biomass was removed, and the filtered nitrogen number is higher than the nitrogen number because, in the absence of biomass, the filtration step enriches for nitrogen rather than removing it. The nitrogen difference was also negative in the aqueous lactic acid stream 971 for fermentation broths B and C and was significantly reduced for fermentation broth A. However, calcium, soluble silica, silica percent volume, and biomass remained high in all processes until after the first separation step after acidulation. In other words, the acidulation station is exposed to calcium, silica, and biomass because there is no efficient and effective removal prior to this point.

### Example 2

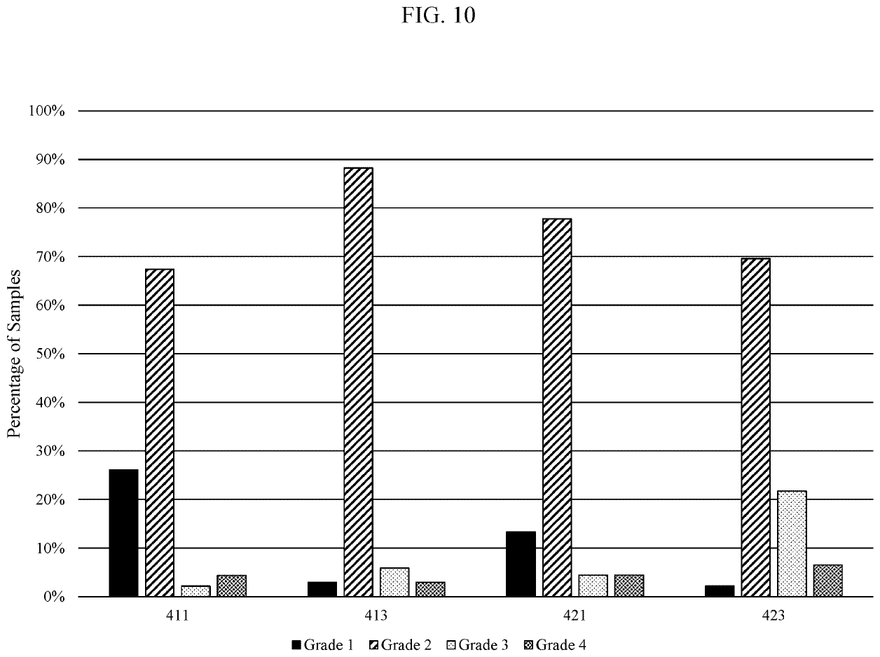

This example demonstrates the compositions of various process intermediates present in the process outlined in FIG. 4. Over a 4-month run of the process outlined in FIG. 4 and described herein, 15 mL samples of the primary hydrocyclone overflow stream 411, the primary hydrocyclone underflow stream 413, the secondary hydrocyclone overflow stream 421, and the secondary hydrocyclone underflow stream 423 were collected twice a day and analyzed for silica content. Following collection, the 15 mL sample was spun down in a centrifuge (spun at 2700 g-force for 5 minutes, on the Hettich Universal 320 centrifuge instrument used this was 4500 rpm) and assigned one of four grades based on the volume of silica present by visual inspection. The grades are outline in Table 3. Table 4 and FIG. 10 report the percentage of sample in each of the four grades for each of the four streams samples. If the primary and secondary hydrocyclones are removing silica from the process, the underflow streams (413 and 423) should have a higher percentage of sample with silica present (grades 2-4) than the overflow streams (411 and 421), which in turn will have a higher percentage of samples with no silica present (grade 1).

TABLE 3

| Sample Grade | Definition |
|---|---|
| 1 | No visual silica present |
| 2 | Less than 0.05 mL silica; less than 0.33% of |
| | the 15 mL sample |
| 3 | Between 0.5 mL and 1.0 mL silica; between 0.33% and |
| | 0.67% of the 15 mL sample |
| 4 | Greater than 1.0 mL silica; greater than 0.67% |
| | of the 15 mL sample |

TABLE 4

Percentage of samples for the indicated
stream with the indicated Grade

| Grade | 411 | 413 | 421 | 423 |
|---|---|---|---|---|
| 1 | 26% | 3% | 13% | 2% |
| 2 | 67% | 88% | 78% | 70% |
| 3 | 2% | 6% | 4% | 22% |
| 4 | 4% | 3% | 4% | 7% |

Results show that 26% of samples in the primary hydrocyclone overflow stream 411 and 13% of samples in the secondary hydrocyclone overflow stream 421 have no silica present (grade 1), demonstrating the success of remove silica using the primary hydrocyclone and secondary hydrocyclones. Likewise, in the primary hydrocyclone underflow stream 413, 88% of samples had less than 0.33% silica, with grades 2, 3, and 4 accounting for a total of 97% of the samples. In the secondary hydrocyclone underflow stream 423, 70% of samples had less than 0.33% silica, with grade 2, 3, and 4 accounting for 98% of the samples. While all four streams sampled show at least 68% of samples with some silica present, the percent of samples without silica is higher in the overflow streams and the percent of samples with silica is highest in the underflow streams, the data shows that the use of the primary and secondary hydrocyclones successfully removes a significant amount of silica from the lactic acid process.

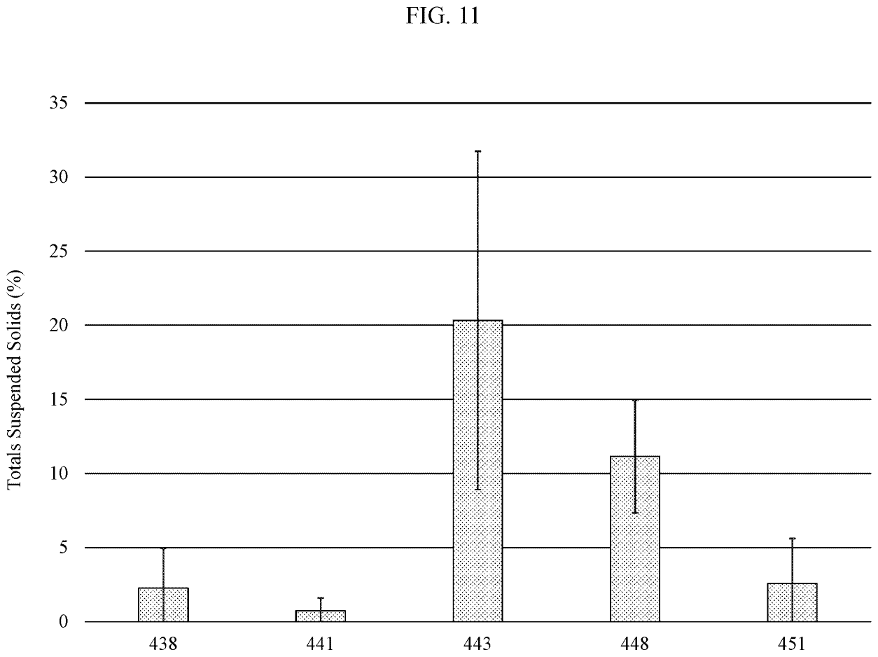

Additionally, over the same 4-month time period, samples were taken from centrifuge feed tank 438, and clarified broth stream 441, the centrifuge concentrate stream 443, the decanter/centrifuge feed collector 448, and the centrate 451. Total suspended solids in each sample were measured by centrifuging each 15 mL sample and assigning a volume of solids visual, reporting the total suspended solids as a volume percentage (% vol). Total suspended solids includes both biomass and silica. Total suspended solids averaged across the samples collected and is reported in Table 5 and FIG. 11.

TABLE 4

| Stream | Total Suspended Solids (% vol) |
|---|---|
| centrifuge feed tank 438 | 2.25 ± 2.70 |
| clarified broth stream 441 | 0.71 ± 0.87 |
| centrifuge concentrate stream 443 | 20.34 ± 11.42 |
| decanter/centrifuge feed collector 448 | 11.14 ± 3.81 |
| centrate 451 | 2.58 ± 3.02 |

The results demonstrate that the centrifuge removes most or all of the biomass and silica present in the decanter/centrifuge feed collector. Removal of the biomass that this stage prevents the biomass and silica from entering the acidulation station 460.

Accordingly, the data in this example demonstrate the effective removal of silica and biomass from the process streams prior to the acidulation station 460. The removal prevents biomass and silica from causing inefficiencies in the acidulation and downstream separation processes and prevents damage to process equipment.

### EXEMPLARY CLAUSES DESCRIBING THE INVENTION

Clause 1. A process for preparing lactic acid comprising:
i) fermenting a carbohydrate source in the presence of a calcium hydroxide-based pH control agent that comprises from about 0.1 to about 10 wt % silica to form a fermentation broth comprising calcium lactate and lactic acid:
ii) directing the fermentation broth to a primary hydrocyclone at a temperature above a temperature at which the calcium lactic is substantially soluble, and withdrawing from the primary hydrocyclone:
   a) a primary hydrocyclone overflow stream comprising lactic acid, calcium lactate, biomass, water, and no more than about 0.05 wt % of silica, and
   b) a primary hydrocyclone underflow stream enriched in silica and comprising water, biomass, lactic acid, and calcium lactate;
iii) directing the primary hydrocyclone underflow stream to a secondary hydrocyclone, and withdrawing from the secondary hydrocyclone:
   c) a secondary hydrocyclone overflow stream comprising lactic acid, calcium lactate, biomass, water, and no more than 0.05 wt % silica, and
   d) a secondary hydrocyclone underflow stream enriched in silica and comprising water, biomass, lactic acid, and calcium lactate;
iv) directing the primary hydrocyclone overflow stream and the secondary hydrocyclone overflow stream to a centrifuge, and withdrawing from the centrifuge:
   e) a clarified broth stream comprising lactic acid and calcium lactate, and
   f) a centrifuge concentrate stream enriched in biomass;
v) directing the clarified broth stream to an acidulation station and acidulating with sulfuric acid to form an aqueous lactic acid product (typically the lactic equivalents present in the aqueous lactic acid product comprising less than ten weight percent (10 wt %) lactate anions, preferably comprising less than five weight percent (5 wt %) lactate anions, more preferably comprising less than one weight percent (1 wt %) lactate anions) and insoluble components comprising gypsum; and
vi) separating the insoluble components comprising gypsum from the aqueous lactic acid product.

Clause 2. A process for preparing lactic acid comprising:
i) fermenting a carbohydrate source in the presence of a calcium hydroxide-based pH control agent that comprises from about 0.1 to about 10 wt % silica to form a fermentation broth comprising lactic acid and calcium lactate:
ii) directing the fermentation broth to a primary hydrocyclone at a temperature above a temperature at which the calcium lactate is substantially soluble, and withdrawing from the primary hydrocyclone:
   a) a primary hydrocyclone overflow stream comprising lactic acid, calcium lactate, biomass, water, and no more than about 0.05 wt % silica, and
   b) a primary hydrocyclone underflow stream enriched in silica and comprising water, biomass, lactic acid, and calcium lactate;
iii) directing the primary hydrocyclone underflow stream to a secondary hydrocyclone and withdrawing from the secondary hydrocyclone:
   c) a secondary hydrocyclone overflow stream comprising lactic acid, calcium lactate, biomass, water, and no more than 0.05 wt % of silica, and
   d) a secondary hydrocyclone underflow stream enriched in silica and comprising water, biomass, lactic acid and calcium lactate;
iv) directing the secondary hydrocyclone overflow stream at a temperature of at least greater than 30° C. (for example from 35° to 45° C. or from 35° to 40° C.), greater than 40°, greater than 50°, or greater than 60° C. to the primary hydrocyclone;
v) directing the primary hydrocyclone overflow stream to a centrifuge and withdrawing from the centrifuge:
   e) a clarified broth stream comprising a lactic acid and calcium lactate, and
   f) a centrifuge concentrate stream enriched in biomass;
vi) directing the clarified broth stream to an acidulation station and acidulating with sulfuric acid to form aqueous lactic acid and insoluble components comprising gypsum; and
vii) separating the insoluble components comprising gypsum from the aqueous lactic acid.

Clause 3. The process of clause 1 or 2, further comprising directing the secondary hydrocyclone underflow stream and the centrifuge concentrate stream enriched in biomass to a mixed biomass/gypsum collector.

Clause 4. The process of clause 3, further comprising directing the insoluble components comprising gypsum to the mixed biomass/gypsum collector.

Clause 5. A process for preparing lactic acid comprising:
i) fermenting a carbohydrate source in the presence of a calcium hydroxide-based pH control agent that comprises from about 0.1 to about 10 wt % silica to form a fermentation broth comprising lactic acid and calcium lactate;
ii) directing the fermentation broth to a primary hydrocyclone at a temperature above a temperature at which the calcium lactate is substantially soluble, and withdrawing from the primary hydrocyclone:
   g) a primary hydrocyclone overflow stream comprising lactic acid, calcium lactate, biomass, water, and no more than about 0.05 wt % of silica, and
   h) a primary hydrocyclone underflow stream enriched in silica and comprising water, biomass, lactic acid and calcium lactate;
iii) directing the primary hydrocyclone overflow stream to a secondary hydrocyclone and withdrawing from the secondary hydrocyclone:
   i) a secondary hydrocyclone overflow stream comprising lactic acid, calcium lactate, biomass, water, and no more than 0.05 wt % of silica, and
   j) a secondary hydrocyclone underflow stream enriched in silica and comprising water, biomass, lactic acid and calcium lactate;
iv) directing the secondary hydrocyclone overflow stream to centrifuge and withdrawing from the centrifuge:
   k) a clarified broth stream comprising lactic acid and calcium lactate, and
   l) a centrifuge concentrate stream enriched in biomass;
v) directing the clarified broth stream to an acidulation station and acidulating with sulfuric acid to form an aqueous lactic acid product (typically the lactic equivalents present in the lactic acid product comprising at least ninety weight percent (90 wt %), typically at least ninety five weight percent (95 wt %) lactic acid, preferably comprising at least ninety nine weight percent (99 wt %) lactic acid) and insoluble components comprising gypsum; and
vi) separating the insoluble components comprising gypsum from the aqueous lactic acid product.

Clause 6. The process of clause 5, further comprising directing the primary hydrocyclone underflow stream, the secondary hydrocyclone underflow stream, and the centrifuge concentrate stream enriched in biomass to a mixed biomass/gypsum collector.

Clause 7. The process of clause 6, further comprising directing the insoluble components comprising gypsum to a mixed biomass/gypsum collector

Clause 8. A process for preparing lactic acid comprising:
i) fermenting a carbohydrate source in the presence of a calcium hydroxide-based pH control agent that comprises from about 0.1 to about 10 wt % silica to form a fermentation broth comprising lactic acid and calcium lactate;
ii) directing the fermentation broth to a primary hydrocyclone at a temperature above a temperature at which the calcium lactate is substantially soluble, and withdrawing from the primary hydrocyclone:
   m) a primary hydrocyclone overflow stream comprising lactic acid, calcium lactate, biomass, water, and no more than about 0.05 wt % of silica, and
   n) a primary hydrocyclone underflow stream enriched in silica and comprising water, biomass, lactic acid, and calcium lactate;
iii) directing the primary hydrocyclone underflow stream to a secondary hydrocyclone and withdrawing from the secondary hydrocyclone:
   o) a secondary hydrocyclone overflow stream comprising lactic acid, calcium lactate, biomass, water, and no more than 0.05 wt % of silica, and
   p) a secondary hydrocyclone underflow stream enriched in silica and comprising water, biomass, lactic acid, and calcium lactate;
iv) directing the primary hydrocyclone overflow stream and the secondary hydrocyclone overflow stream to a centrifuge and withdrawing from the centrifuge:
   q) a clarified broth stream comprising lactic acid and calcium lactate, and
   r) a centrifuge concentrate stream enriched in biomass;
v) directing the secondary hydrocyclone underflow stream and the centrifuge concentrate stream enriched in biomass to a decanter/centrifuge as a decanter/centrifuge biomass feed having a combined dry substance content of from about 1 to about 30 wt %, and withdrawing from the decanter/centrifuge
   s) a decanter/centrifuge centrate comprising lactic acid and calcium lactate, and
   t) a decanter/centrifuge underflow stream enriched in biomass having a dry substance content of at least about 35 wt %;
vi) directing the clarified broth stream and the decanter/centrifuge centrate to an acidulation station and acidulating with sulfuric acid to form an aqueous lactic acid product (typically the lactic equivalents present in the lactic acid product comprising less than five weight percent (5 wt %) lactate anions, preferably comprising less than one weight percent (1 wt %) lactate anions) and insoluble components comprising gypsum; and
v) separating the insoluble components comprising gypsum from the aqueous lactic acid product.

Clause 9. A process for preparing lactic acid comprising:
i) fermenting a carbohydrate source in the presence of a calcium hydroxide-based pH control agent that comprises from about 0.1 to about 10 wt % silica to form a fermentation broth comprising lactic acid and calcium lactate;
ii) directing the fermentation broth to a primary hydrocyclone at a temperature above a temperature at which the calcium lactate is substantially soluble, and withdrawing from the primary hydrocyclone:
   u) a primary hydrocyclone overflow stream comprising lactic acid, calcium lactate, biomass, water, and no more than about 0.05 wt % of silica, and
   v) a primary hydrocyclone underflow stream enriched in silica and comprising water, biomass, lactic acid, and calcium lactate;
iii) directing the primary hydrocyclone underflow stream to a secondary hydrocyclone and withdrawing from the secondary hydrocyclone:
   w) a secondary hydrocyclone overflow stream comprising lactic acid, calcium lactate, biomass, water, and no more than 0.05 wt % of silica, and
   x) a secondary hydrocyclone underflow stream enriched in silica and comprising water, biomass, lactic acid, and calcium lactate;
iv) directing the secondary hydrocyclone overflow stream at a temperature of greater than 30° C. (for example from 35° to 45° C. or from 35° to 40° C.), greater than 40°, greater than 50°, or greater than 60° C. to the primary hydrocyclone;
v) directing the primary hydrocyclone overflow stream to a centrifuge and withdrawing from the centrifuge:
   y) a clarified broth stream comprising lactic acid and calcium lactate, and
   z) a centrifuge concentrate stream enriched in biomass;
vi) directing the secondary hydrocyclone underflow stream and the centrifuge concentrate stream enriched in biomass to a decanter/centrifuge as a decanter/centrifuge biomass feed having a dry substance content of from about 1 to about 30 wt %, and withdrawing from the decanter/centrifuge
   aa) a decanter/centrifuge centrate comprising lactic acid and calcium lactate, and
   bb) a decanter/centrifuge underflow stream enriched in biomass having a dry substance content of at least about 35 wt %;
vii) directing the clarified broth stream and the decanter/centrifuge centrate to an acidulation station and acidulating with sulfuric acid to form an aqueous lactic acid product (typically the lactic equivalents present in the lactic acid product comprising less than five weight percent (5 wt %) lactate anions, preferably comprising less than one weight percent (1 wt %) lactate anions) and insoluble components comprising gypsum; and
viii) separating the insoluble components comprising gypsum from the aqueous lactic acid product.

Clause 10. A process for preparing lactic acid comprising:
i) fermenting a carbohydrate source in the presence of a calcium hydroxide-based pH control agent that comprises from about 0.1 to about 10 wt % silica to form a fermentation broth comprising lactic acid and calcium lactate;
ii) directing the fermentation broth to a primary hydrocyclone at a temperature above a temperature at which the calcium lactate is substantially soluble, and withdrawing from the primary hydrocyclone:
   cc) a primary hydrocyclone overflow stream comprising lactic acid, calcium lactate, biomass, water, and no more than about 0.05 wt % of silica, and
   dd) a primary hydrocyclone underflow stream enriched in silica and comprising water, biomass, lactic acid, and calcium lactate;
iii) directing the primary hydrocyclone overflow stream to a secondary hydrocyclone and withdrawing from the secondary hydrocyclone:
   ee) a secondary hydrocyclone overflow stream comprising lactic acid, calcium lactate, biomass, water, and no more than 0.05 wt % of silica, and
   ff) a secondary hydrocyclone underflow stream enriched in silica and comprising water, biomass, lactic acid, and calcium lactate;
iv) directing the secondary hydrocyclone overflow stream to a centrifuge that separates the hydrocyclone overflow stream into
   gg) a clarified broth stream comprising lactic acid and calcium lactate, and
   hh) a centrifuge concentrate stream enriched in biomass:
v) directing the primary hydrocyclone underflow stream, the secondary hydrocyclone underflow stream, and the centrifuge concentrate stream enriched in biomass as a decanter/centrifuge biomass feed having a dry substance content of from about 1 to about 30 wt %, and withdrawing from the decanter/centrifuge
   ii) a decanter/centrifuge centrate comprising lactic acid and calcium lactate, and
   jj) a decanter/centrifuge underflow stream enriched in biomass having a dry substance content of at least about 35 wt %;
vi) directing the clarified broth stream and the decanter/centrifuge centrate to an acidulation station and acidulating with sulfuric acid to form an aqueous lactic acid product (typically the lactic equivalents present in the lactic acid product comprising less than five weight percent (5 wt %) lactate anions, preferably comprising less than one weight percent (1 wt %) lactate anions) and insoluble components comprising gypsum; and
vii) separating the insoluble components comprising gypsum from the aqueous lactic acid product.

Clause 11. The process of any one of clauses 8-10, further comprising directing the decanter/centrifuge underflow stream enriched in biomass and the insoluble components comprising gypsum to a mixed biomass/gypsum collector.

Clause 12. The process of any one of clauses 1-11, wherein the fermentation is carried out using yeast; or wherein the fermentation is carried out using bacteria.

Clause 13. The process of any one of clauses 1-12, wherein the calcium hydroxide-based pH control agent has a silica content of from about 1.5 to about 8.5 wt % silica; or wherein the calcium hydroxide-based pH control agent has a silica content of from about 1.5 to 7.5 wt %; or wherein the calcium hydroxide-based pH control agent has a silica content of from about 2.5 to 7.5 wt % silica; or wherein the calcium hydroxide-based pH control agent has a silica content of from about 3 to about 7 wt % silica.

Clause 14. The process of any one of clauses 1-13, further comprising reducing the liquid content of the fermentation broth by a broth evaporator prior to directing the fermentation broth to the primary hydrocyclone.

Clause 15. The process of clause 14, wherein the total lactic acid and calcium lactate concentration at the inlet to the primary hydrocyclone is at least about 150 g/L of lactic equivalents; or wherein the total lactic acid and calcium lactate concentration at the inlet to the primary hydrocyclone is at least about 200 g/L of lactic equivalents; or wherein the total lactic acid and calcium lactate concentration at the inlet to the primary hydrocyclone is at least about 250 g/L of lactic equivalents, or wherein the total lactic acid and calcium lactate concentration at the inlet to the primary hydrocyclone is at least about 300 g/L of lactic equivalents; or wherein the total lactic acid and calcium lactate concentration at the inlet to the primary hydrocyclone is from about 200 g/L of lactic equivalents to about 350 g/L of lactic equivalents; or wherein the total lactic acid and calcium lactate concentration at the inlet to the primary hydrocyclone is from about 200 g/L of lactic equivalents to about 330 g/L of lactic equivalents; or wherein the total lactic acid and calcium lactate concentration at the inlet to the primary hydrocyclone is from about 200 g/L of lactic equivalents to about 300 g/L of lactic equivalents; or wherein the total lactic acid and calcium lactate concentration at the inlet to the primary hydrocyclone is from about 250 g/L of lactic equivalents to about 350 g/L of lactic equivalents; or wherein the total lactic acid and calcium lactate concentration at the inlet to the primary hydrocyclone is from about 250 g/L of lactic equivalents to about 330 g/L of lactic equivalents; or wherein the total lactic acid and calcium lactate concentration at the inlet to the primary hydrocyclone is from about 250 g/L of lactic equivalents to about 300 g/L of lactic equivalents.

Clause 16. The process of any one of clauses 1-15, wherein the fermentation broth is directed to the primary hydrocyclone at a temperature wherein at least 90 wt % of the calcium lactate present is solubilized; or wherein the fermentation broth is directed to the primary hydrocyclone at a temperature wherein at least 95 wt % of the calcium lactate present is solubilized; or wherein the fermentation broth is directed to the primary hydrocyclone at a temperature wherein at least 97 wt % of the calcium lactate present is solubilized; or wherein the fermentation broth is directed to the primary hydrocyclone at a temperature wherein at least 98 wt % of the calcium lactate present is solubilized; or wherein the fermentation broth is directed to the primary hydrocyclone at a temperature wherein at least 99 wt % of the calcium lactate present is solubilized.

Clause 17. The process of any one of clauses 1-16, wherein the fermentation broth is directed to the primary hydrocyclone at a temperature of at least 30° C. (for example, from 35° C. to 45° C. or from 35° C. to 40° C.), at least 40° C., at least 50° C. or at least 60° C.

Clause 18. The process of any one of clauses 1-17, wherein the fermentation broth is directed to the primary hydrocyclone at a temperature of from about 60° C. to about 85° C.; or wherein the fermentation broth is directed to the primary hydrocyclone at a temperature of from about 70° C. to about 80° C.

Clause 19. The process of any one of clauses 1-18, wherein any of the streams comprising calcium lactate are maintained at a temperature and calcium lactate concentration throughout the process such that at least 90 wt % of the calcium lactate present is solubilized; or wherein any of the streams comprising calcium lactate are maintained at a temperature and calcium lactate concentration throughout the process such that at least 95 wt % of the calcium lactate present is solubilized; or wherein any of the streams comprising calcium lactate are maintained at a temperature and calcium lactate concentration throughout the process such that at least 97 wt % of the calcium lactate present is solubilized; or wherein any of the streams comprising calcium lactate are maintained at a temperature and calcium lactate concentration throughout the process such that at least 98 wt % of the calcium lactate present is solubilized; or wherein any of the streams comprising calcium lactate are maintained at a temperature and calcium lactate concentration throughout the process such that at least 99 wt % of the calcium lactate present is solubilized.

Clause 20. The process of any one of clauses 1-19, wherein the primary hydrocyclone comprises a plurality of hydrocyclones arranged in parallel.

Clause 21. The process of any one of clauses 1-20, wherein the secondary hydrocyclone comprises a plurality of hydrocyclones arranged in parallel.

Clause 22. The process of any one of clauses 1-21, wherein the primary hydrocyclone is operated under an initial pressure of at least 5 psig, at least 10 psig, at least 15 psig, at least 20 psig, at least 25 psig, or at least 30 psig.

Clause 23. The process of any one of clauses 1-22, wherein the secondary hydrocyclone is operated under an initial pressure of at least 5 psig, at least 10 psig, at least 15 psig, at least 20 psig, at least 25 psig, or at least 30 psig.

Clause 24. The process of any one of clauses 1-23, wherein flow through the primary hydrocyclone and/or the secondary hydrocyclone is regulated by valving cyclones on and off, and not by reducing feed pressure via a control valve.

Clause 25. The process of any one of clauses 1-24, wherein the primary hydrocyclone and/or the secondary hydrocyclone is provided with a strainer upstream of the hydrocyclone that is in turn configured for automatic periodic flush of the strainer, thereby preventing clogging of the strainer.

Clause 26. The process of any one of clauses 1 and 8 wherein the primary hydrocyclone overflow stream and the secondary hydrocyclone overflow stream are directed to a centrifuge feed tank for mixing prior to being directed to the centrifuge.

Clause 27. The process of any one of clauses 1-26, wherein the centrifuge comprises a plurality of centrifuges arranged in parallel.

Clause 28. The process of any one of clauses 8 and 9, wherein the secondary hydrocyclone underflow stream and the biomass enriched concentrate stream are combined together by metering each stream directly into the decanter/centrifuge as a decanter/centrifuge biomass feed.

Clause 29. The process of any one of clauses 8 and 9, wherein the secondary hydrocyclone underflow stream and the biomass enriched concentrate stream are combined together by directing each stream into a decanter/centrifuge and biomass feed collector for mixing and directing the resulting mixture to the decanter/centrifuge as a decanter/centrifuge biomass feed.

Clause 30. The process of clause 10, wherein the primary hydrocyclone underflow stream, the secondary hydrocyclone underflow stream, and the biomass enriched concentrate stream are combined together by metering each stream directly into the decanter/centrifuge as a decanter/centrifuge biomass feed.

Clause 31. The process of clause 10, wherein the primary hydrocyclone underflow stream, the secondary hydrocyclone underflow stream, and the biomass enriched concentrate stream are combined together by directing each stream into a decanter/centrifuge and biomass feed collector for mixing and directing the resulting mixture to the decanter/centrifuge as a decanter/centrifuge biomass feed.

Clause 32. The process of any one of clauses 28-31, wherein the decanter/centrifuge biomass feed has a dry substance content of from about 2 to about 20 wt %, or wherein the decanter/centrifuge biomass feed has a dry substance content of from about 4 to about 15 wt %, or wherein the decanter/centrifuge biomass feed has a dry substance content of from about 5 to about 10 wt %.

Clause 33. The process of any one of clauses 8-10, wherein flow of feed streams to the decanter/centrifuge is controlled such that the decanter/centrifuge is operated at no less than half of maximum operational rate of the centrifuge, thereby maintaining an acceptable high concentration of the biomass enriched concentrate stream that in turn maintains an appropriate decanter/centrifuge biomass feed dry substance content for operation of the decanter/centrifuge.

Clause 34. The process of any one of clauses 8-10, wherein the decanter/centrifuge comprises a bowl configured with a side ejection port to purge the biomass enriched concentrate stream from the bowl.

Clause 35. The process of clause 34, wherein the decanter/centrifuge comprises a set of interchangeable bowls such that one bowl can be removed and rapidly exchanged with another bowl.

Clause 36. The process of any one of clauses 8-10, wherein the decanter/centrifuge comprises a plurality of decanter/centrifuges arranged in parallel.

Clause 37. The process of any one of clauses 1-36, further comprising treating the aqueous lactic acid product to provide an aqueous lactic acid product having a lactic acid concentration of at least 60 wt %; further comprising treating the aqueous lactic acid product to provide an aqueous lactic acid product having a lactic acid concentration of at least 70 wt %, or further comprising treating the aqueous lactic acid product to provide an aqueous lactic acid product having a lactic acid concentration of at least 80 wt %; further comprising treating the aqueous lactic acid product to provide an aqueous lactic acid product having a lactic acid concentration of at least 85 wt %; or further comprising treating the aqueous lactic acid product to provide an aqueous lactic acid product having a lactic acid concentration of at least 90 wt %; or further comprising treating the aqueous lactic acid product to provide an aqueous lactic acid product having a lactic acid concentration of at least 95 wt %.

Clause 38. The process of clause 37, wherein the treating the aqueous lactic acid product step comprises using a distillation process to increase the lactic acid concentration.

Clause 39. The process of clause 38, wherein the distillation process comprises the use of a wiped film evaporator; or wherein the distillation process comprises the use of a distillation column; or wherein the distillation process comprises the use of a boiling tube evaporator.

Clause 40. A process for recovering lactic acid through a gypsum removal process, the process comprising the steps of
a) obtaining a fermentation broth comprising:
   i) lactic acid product having from about 100 g/L to about 150 g/L of lactic equivalents; and
   ii) a calcium hydroxide-based pH control agent that comprises from about 0.1 to about 10 wt % silica;
b) evaporating the fermentation broth to increase the lactic acid product concentration to from 200 g/L to 350 g/L lactic equivalents, or to from 250 g/L to 330 g/L lactic equivalents;
c) processing the evaporated broth with at least one hydrocyclone to produce:
   (iii) a hydrocyclone overflow stream comprising lactic acid, calcium lactate, biomass, water, and no more than about 0.05 wt % silica and
   (iv) a hydrocyclone underflow stream enriched in silica and comprising water, biomass, calcium lactate and lactic acid;
d) directing the hydrocyclone overflow stream to a centrifuge and withdrawing from the centrifuge:
   (v) a clarified broth stream comprising lactic acid and soluble calcium lactate and
   (vi) a centrifuge concentrate stream enriched in biomass:
e) acidulating the clarified broth stream to produce aqueous lactic acid and gypsum; and
f) separating gypsum from the aqueous lactic acid.

Clause 41. The process of clause 40, further comprising g) treating the aqueous lactic acid to increase the lactic acid concentration to at least 60 wt %, or treating the aqueous lactic acid to increase the lactic acid concentration to at least 70 wt %, or treating the aqueous lactic acid to increase the lactic acid concentration to at least 80 wt %, or treating the aqueous lactic acid to increase the lactic acid concentration to at least wt %, or treating the aqueous lactic acid to increase the lactic acid concentration to at least 90 wt %, or treating the aqueous lactic acid to increase the lactic acid concentration to at least 95 wt %.

Clause 42. The process of clause 41, wherein step g) comprises using a distillation process to increase the lactic acid concentration.

Clause 43. The process of any of clauses 40 through 42, wherein the calcium hydroxide-based pH control agent comprises from 1.5 to 8.5 wt % silica.

Clause 44. The process of any of clauses 40 through 42, wherein the calcium hydroxide-based pH control agent comprises from 1.5 to 7.5 wt % silica.

Clause 45. The process of any of clauses 40 through 42, wherein the calcium hydroxide-based pH control agent comprises from 2.5 to 7.5 wt % silica.

Clause 46. The process of any of clauses 40 through 42, wherein the calcium hydroxide-based pH control agent comprises from 3.0 to 7.0 wt % silica.

Clause 47. The process of any of clauses 1 through 46, wherein the process is a continuous process.

Clause 48. The process of clause 47, wherein the process is operated continuously for at least four (4) days.

Clause 49. The process of clause 47, wherein the process is operated continuously for at least five (5) days.

Clause 50. The process of clause 47, wherein the process is operated continuously for at least six (6) days.

Clause 51. The process of clause 47, wherein the process is operated continuously for at least seven (7) days.

Clause 52. The process of clause 47, wherein the process is operated continuously for at least fourteen (14) days.

Clause 53. The process of clause 47, wherein the process is operated continuously for at least thirty (30) days.

Clause 54. The process of any of clauses 40 through 53, wherein the evaporated broth is at a temperature of at least 50° C., at least 60° C., at least 70° C. (for example from 50° C. to 90° C., from 60° C. to 85° C., or from 70° C. to 80° C.) after evaporation and prior to acidulation.

Clause 55. The process of any of clause 1 through 39, wherein the fermentation broth is evaporated to increase the lactic acid product concentration to from 200 g/L to 350 g/L lactic equivalents, or to from 250 g/L to 330 g/L lactic equivalents prior to directing the fermentation broth to the primary hydrocyclone.

Clause 56. The process of any of clause 1 through 39, wherein the fermentation broth is evaporated to increase the lactic acid product concentration to from 10 wt % to 30 wt % (for example from 15 wt % to 25 wt % or from 18 wt % to 23 wt %) prior to directing the fermentation broth to the primary hydrocyclone.

Clause 57. The process of any of clauses 55 and 56, wherein the evaporated fermentation broth is maintained at a temperature of at least 50° C., at least 60° C., at least 70° C. (for example from 50° C. to 90° C., from 60° C. to 85° C., or from 70° C. to 80° C.) after evaporation and prior to acidulation.

Clause 58. The process of any of clauses 1-57, wherein calcium lactate present is substantially soluble during the process.

Clause 59. The process of any of clauses 1-39, wherein calcium lactate in the fermentation broth directed to the primary hydrocyclone is substantially soluble (for example, at last 95 wt % is soluble, at least 96 wt %, at least 97 wt %, at least 98 wt %, at least 99 wt % is soluble).

Clause 60. The process of any of clause 40-54, and 58, wherein calcium lactate in the evaporated broth processed with at least one hydrocyclone is substantially soluble (for example, at last 95 wt % is soluble, at least 96 wt %, at least 97 wt %, at least 98 wt %, at least 99 wt % is soluble).

Clause 61. The process of any of clauses 40 through 54 and 60, wherein the at least one hydrocyclone comprises a primary hydrocyclone and a secondary hydrocyclone and wherein:
a hydrocvclone underflow stream from the primary hydrocyclone is directed to the secondary hydrocyclone; and
a hydrocyclone overflow stream from the primary hydrocyclone is directed to the centrifuge.

Clause 62. The process of clause 61, wherein:
a hydrocyclone overflow stream from the secondary hydrocyclone is directed to the centrifuge or the inlet of the primary hydrocyclone.

Clause 63. The process of any of clause 61 and 62, wherein:
a primary hydrocyclone underflow stream and a secondary hydrocyclone underflow stream are directed to a decanter/centrifuge biomass feed (optionally having a dry substance content of from about 1 to about 30 wt %), and withdrawing from the decanter/centrifuge:
a decanter/centrifuge centrate comprising lactic acid and calcium lactate, and
a decanter/centrifuge underflow stream enriched in biomass (optionally having a dry substance content of at least 35 wt %).

Clause 64. The process of any of clause 61 and 62, wherein:
a primary hydrocyclone underflow stream is directed to a decanter/centrifuge biomass feed (optionally having a dry substance content of from about 1 to about 30 wt %), and withdrawing from the decanter/centrifuge:
a decanter/centrifuge centrate comprising lactic acid and calcium lactate, and
a decanter/centrifuge underflow stream enriched in biomass (optionally having a dry substance content of at least about 35 wt %).

Clause 65. The process of any of clauses 63 and 64, wherein:
the decanter/centrifuge centrate together with the clarified broth stream are directed to an acidulation station and acidulated with sulfuric acid to form an aqueous lactic acid product comprising less than five weight percent (5 wt %) lactate anions (for example comprising less than one weight percent (1 wt %) lactate anions) and insoluble components comprising gypsum; and
separating the insoluble components comprising gypsum from the aqueous lactic acid product.

Clause 66. The process of any of clauses 61 and 62, wherein the secondary hydrocyclone underflow stream and the centrifuge concentrate stream enriched in biomass are directed to a mixed biomass/gypsum collector.

Clause 67. The process of clause 66, further comprising directing insoluble components comprising gypsum to the mixed biomass/gypsum collector.

1. A process for recovering lactic acid through a gypsum removal process, the process comprising the steps of:
directing a fermentation broth comprising lactic acid to at least one hydrocyclone to produce:
a hydrocyclone overflow stream comprising lactic acid, calcium lactate, biomass, water, and no more than about 0.05 wt % silica; and
a hydrocyclone underflow stream enriched in silica and comprising water, biomass, calcium lactate and lactic acid;
directing the hydrocyclone overflow stream to a centrifuge and withdrawing from the centrifuge:
a clarified broth stream comprising lactic acid and soluble calcium lactate; and
a centrifuge concentrate stream enriched in biomass;
acidulating the clarified broth stream to produce aqueous lactic acid and gypsum; and
separating gypsum from the aqueous lactic acid product.

2. The process for preparing lactic acid of claim 1, wherein the process comprises:
i) fermenting a carbohydrate source in the presence of a calcium hydroxide-based pH control agent that comprises from about 0.1 to about 10 wt % silica to form a fermentation broth comprising lactic acid and calcium lactate;
ii) directing the fermentation broth to a primary hydrocyclone at a temperature above a temperature at which the calcium lactate is substantially soluble, and withdrawing from the primary hydrocyclone:
a) a primary hydrocyclone overflow stream comprising lactic acid, calcium lactate, biomass, water, and no more than about 0.05 wt % silica, and
b) a primary hydrocyclone underflow stream enriched in silica and comprising water, biomass, lactic acid, and calcium lactate;
iii) directing the primary hydrocyclone underflow stream to a secondary hydrocyclone and withdrawing from the secondary hydrocyclone:
c) a secondary hydrocyclone overflow stream comprising lactic acid, calcium lactate, biomass, water, and no more than 0.05 wt % of silica, and
d) a secondary hydrocyclone underflow stream enriched in silica and comprising water, biomass, lactic acid and calcium lactate;
iv) directing the primary hydrocyclone overflow stream and the secondary hydrocyclone overflow stream to a centrifuge and withdrawing from the centrifuge:
e) a clarified broth stream comprising a lactic acid and calcium lactate, and
f) a centrifuge concentrate stream enriched in biomass;
v) directing the clarified broth stream to an acidulation station and acidulating with sulfuric acid to form aqueous lactic acid and insoluble components comprising gypsum; and
vi) separating the insoluble components comprising gypsum from the aqueous lactic acid product.

3. The process for preparing lactic acid of claim 1, wherein the process comprises:
i) fermenting a carbohydrate source in the presence of a calcium hydroxide-based pH control agent that comprises from about 0.1 to about 10 wt % silica to form a fermentation broth comprising lactic acid and calcium lactate;
ii) directing the fermentation broth to a primary hydrocyclone at a temperature above a temperature at which the calcium lactate is substantially soluble, and withdrawing from the primary hydrocyclone:
a) a primary hydrocyclone overflow stream comprising lactic acid, calcium lactate, biomass, water, and no more than about 0.05 wt % silica, and
b) a primary hydrocyclone underflow stream enriched in silica and comprising water, biomass, lactic acid, and calcium lactate;
iii) directing the primary hydrocyclone underflow stream to a secondary hydrocyclone and withdrawing from the secondary hydrocyclone:
c) a secondary hydrocyclone overflow stream comprising lactic acid, calcium lactate, biomass, water, and no more than 0.05 wt % of silica, and
d) a secondary hydrocyclone underflow stream enriched in silica and comprising water, biomass, lactic acid and calcium lactate;
iv) directing the secondary hydrocyclone overflow stream at a temperature of at least greater than 30° C. (for example from 35° to 45° C. or from 35° to 40° C.), greater than 40°, greater than 50°, or greater than 60° C. to the primary hydrocyclone;
v) directing the primary hydrocyclone overflow stream to a centrifuge and withdrawing from the centrifuge:
e) a clarified broth stream comprising a lactic acid and calcium lactate, and
f) a centrifuge concentrate stream enriched in biomass;
vi) directing the clarified broth stream to an acidulation station and acidulating with sulfuric acid to form aqueous lactic acid and insoluble components comprising gypsum; and
vii) separating the insoluble components comprising gypsum from the aqueous lactic acid product.

4. The process for preparing lactic acid of claim 1, wherein the process comprises:
i) fermenting a carbohydrate source in the presence of a calcium hydroxide-based pH control agent that comprises from about 0.1 to about 10 wt % silica to form a fermentation broth comprising lactic acid and calcium lactate;
ii) directing the fermentation broth to a primary hydrocyclone at a temperature above a temperature at which the calcium lactate is substantially soluble, and withdrawing from the primary hydrocyclone:
a) a primary hydrocyclone overflow stream comprising lactic acid, calcium lactate, biomass, water, and no more than about 0.05 wt % of silica, and
b) a primary hydrocyclone underflow stream enriched in silica and comprising water, biomass, lactic acid and calcium lactate;
iii) directing the primary hydrocyclone overflow stream to a secondary hydrocyclone and withdrawing from the secondary hydrocyclone:
c) a secondary hydrocyclone overflow stream comprising lactic acid, calcium lactate, biomass, water, and no more than 0.05 wt % of silica, and
d) a secondary hydrocyclone underflow stream enriched in silica and comprising water, biomass, lactic acid and calcium lactate;
iv) directing the secondary hydrocyclone overflow stream to centrifuge and withdrawing from the centrifuge:
e) a clarified broth stream comprising lactic acid and calcium lactate, and
f) a centrifuge concentrate stream enriched in biomass;
v) directing the clarified broth stream to an acidulation station and acidulating with sulfuric acid to form an aqueous lactic acid product and insoluble components comprising gypsum; and
vi) separating the insoluble components comprising gypsum from the aqueous lactic acid product.

5. The process of claim 1 wherein the fermentation broth comprises from about 100 g/L to about 150 g/L of lactic equivalents and the process comprises evaporating the fermentation broth to increase the lactic acid product concentration to 200 g/L to 350 g/L lactic equivalents or 250 g/L to 330 g/L lactic equivalents and directing the evaporated fermentation broth to the one or more hydrocyclones.

6. The process of claim 1 additionally comprising
directing the hydrocyclone underflow stream (e.g., the primary hydrocyclone underflow stream and/or the secondary hydrocyclone underflow stream) and the centrifuge concentrate stream enriched in biomass to a decanter/centrifuge as a decanter/centrifuge feed stream having a combined dry substance content of from about 1 to about 30 wt %, and withdrawing from the decanter/centrifuge
a) a decanter/centrifuge centrate comprising lactic acid and calcium lactate, and
b) a decanter/centrifuge underflow stream enriched in biomass having a dry substance content of at least about 35 wt %;
and directing the decanter/centrifuge centrate to the acidulation station.

7. The process of claim 2, further comprising:
directing the secondary hydrocyclone underflow stream; the centrifuge concentrate stream enriched in biomass; and/or the insoluble components stream to a mixed biomass/gypsum collector; and/or
directing the decanter/centrifuge underflow stream enriched in biomass and the insoluble components comprising gypsum to a mixed biomass/gypsum collector.

8. The process of claim 1 wherein the lactic equivalents present in the lactic acid product comprising at least ninety weight percent (90 wt %), typically at least ninety five weight percent (95 wt %) lactic acid, preferably comprising at least ninety nine weight percent (99 wt %) lactic acid.

9. (canceled)

10. The process of claim 1 wherein the calcium hydroxide-based pH control agent has a silica content of from about 1.5 to about 8.5 wt %, from about 1.5 to 7.5 wt %, from about 2.5 to 7.5 wt %, or from about 3 to about 7 wt % silica.

11. The process of claim 1 wherein the total lactic acid and calcium lactate concentration at the inlet to the one or more hydrocyclones (e.g., primary hydrocyclone and/or secondary hydrocyclone) is at least about 150 g/L, at least about 250 g/L, at least about 300 g/L, from about 200 g/L to about 350 g/L, from about 200 g/L to about 330 g/L, from about 200 g/L to about 300 g/L, from about 250 g/L to about 350 g/L, from about 250 g/L to about 330 g/L, or from about 250 g/L of lactic equivalents to about 300 g/L of lactic equivalents.

12. (canceled)

13. (canceled)

14. The process of claim 1, wherein the fermentation broth is directed to the one or more hydrocyclones (e.g., primary hydrocyclone and/or secondary hydrocyclone) at a temperature of from about 60° C. to about 85° C. or from about 70° C. to about 80° C.

15. The process of claim 1 wherein any of the streams comprising calcium lactate are maintained throughout the process at a temperature and calcium lactate concentration such that at least 90 wt %, at least 95 wt %, at least 97 wt %, at least 98 wt %, or at least 99 wt % of the calcium lactate present is solubilized.

16. The process of claim 1, wherein the one or more hydrocyclones (e.g., primary hydrocyclone and/or secondary hydrocyclone) comprises a plurality of hydrocyclones arranged in parallel.

17. The process of claim 1, wherein one or more hydrocyclones (e.g., primary hydrocyclone and/or secondary hydrocyclone) is operated under an initial pressure of at least 5 psig, at least 10 psig, at least 15 psig, at least 20 psig, at least 25 psig, or at least 30 psig.

18. The process of claim 1 wherein flow through the one or more hydrocyclones (e.g., the primary hydrocyclone and/or the secondary hydrocyclone) is regulated by valving cyclones on and off, and not by reducing feed pressure via a control valve.

19. The process of claim 1 wherein one or more hydrocyclones (e.g., primary hydrocyclone and/or secondary hydrocyclone) is provided with a strainer upstream of the hydrocyclone that is in turn configured for automatic periodic flush of the strainer, thereby preventing clogging of the strainer.

20. The process of claim 1 wherein the process comprises a centrifuge feed tank for mixing multiple feed streams prior to being directed to the centrifuge.

21. (canceled)

22. The process of claim 6,
wherein the hydrocyclone underflow stream (e.g., primary hydrocyclone underflow stream and/or secondary hydrocyclone underflow stream) and the centrifuge concentrate stream are combined together by metering each stream directly into the decanter/centrifuge as a decanter/centrifuge feed stream; or
wherein the hydrocyclone underflow stream (e.g., primary hydrocyclone underflow stream and/or secondary hydrocyclone underflow stream) and the centrifuge concentrate stream are combined together by directing each stream into a decanter/centrifuge feed collector for mixing and directing the resulting mixture to the decanter/centrifuge as a decanter/centrifuge feed stream.

23. The process of claim 6, wherein the decanter/centrifuge feed stream has a dry substance content of from about 2 to about 20 wt %, from about 4 to about 15 wt %, or from about 5 to about 10 wt %.

24. The process of claim 6, wherein flow of feed streams to the decanter/centrifuge is controlled such that the decanter/centrifuge is operated at no less than half of maximum operational rate of the centrifuge.

25. The process of claim 6, wherein the decanter/centrifuge comprises
a bowl configured with a side ejection port to purge the biomass enriched concentrate stream from the bowl;
a set of interchangeable bowls such that one bowl can be removed and rapidly exchanged with another bowl; and/or
a plurality of decanter/centrifuges arranged in parallel.

26. (canceled)

27. (canceled)

28. (canceled)

29. (canceled)

30. The process of claim 1 wherein the fermentation broth is evaporated to increase the lactic acid product concentration to 200 g/L to 350 g/L lactic equivalents, or 250 g/L to 330 g/L lactic equivalents prior to directing the fermentation broth to the primary hydrocyclone; and/or
wherein the fermentation broth is evaporated to increase the lactic acid product concentration to 10 wt % to 30 wt % (for example from 15 wt % to 25 wt % or from 18 wt % to 23 wt %) prior to directing the fermentation broth to the primary hydrocyclone.

31. A centrifuge concentrate composition enriched in biomass produced by claim 1, wherein the composition comprises 1 wt % to 30 wt % biomass, silica, and calcium.

32. A decanter/centrifuge underflow composition produced by claim 1 wherein the composition comprises at least 30 wt % or at least 35 wt % biomass, silica, and calcium.

33. (canceled)
